(19) United States
(12) Patent Application Publication
(10) Pub. No.: US 2023/0137630 A1
(43) Pub. Date: May 4, 2023

(54) RECOMBINANT MICROALGAE ABLE TO PRODUCE KTTKS PEPTIDES, POLYPEPTIDES, OR PROTEINS AND THEIR DERIVATIVES AND ASSOCIATED METHOD AND USES THEREOF

(71) Applicants: Sederma, Le Perray-en-Yvelines (FR); Alganelle, Le Bourget du Lac (FR)

(72) Inventor: Ghislaine Tissot-Lecuelle, La Motte Servolex (FR)

(73) Assignees: Sederma, Le Perray-en-Yvelines (FR); Alganelle, Le Bourget du Lac (FR)

(21) Appl. No.: 17/802,572

(22) PCT Filed: Feb. 25, 2021
(86) PCT No.: PCT/EP2021/054749

§ 371 Date: Aug. 26, 2022

(30) Foreign Application Priority Data
Feb. 28, 2020 (FR) 2002027

(51) Int. Cl. C07K 7/06; A61K 8/64; A61Q 19/08; C12N 15/79; C07K 14/745

(52) U.S. Cl. CPC C07K 7/06; A61K 8/64; A61Q 19/08; C12N 15/79; C07K 14/745; C07K 2319/02

(57) ABSTRACT

The present invention concerns a recombinant microalgae comprising a nucleic acid sequence encoding a recombinant peptide of KTTKS (SEQ ID No 1); a recombinant peptide, polypeptide or protein consisting in repeated units of SEQ ID No 1; or a derivative thereof, said nucleic acid sequence being located in the chloroplast genome of microalgae. It also relates to a method for producing a recombinant peptide of SEQ ID No 1; a recombinant peptide, polypeptide or protein consisting in repeated units of SEQ ID No 1; or a derivative thereof, wherein said method comprises the chloroplast genome transformation of a microalgae with a nucleic acid sequence encoding said recombinant protein, polypeptide or peptide. It further relates to the use of said recombinant peptide, polypeptide or protein for the cosmetic industry.

The present invention concerns a recombinant microalgae comprising a nucleic acid sequence encoding a recombinant peptide of KTTKS (SEQ ID No 1); a recombinant peptide, polypeptide or protein consisting in repeated units of SEQ ID No 1; or a derivative thereof, said nucleic acid sequence being located in the chloroplast genome of microalgae. It also relates to a method for producing a recombinant peptide of SEQ ID No 1; a recombinant peptide, polypeptide or protein consisting in repeated units of SEQ ID No 1; or a derivative thereof, wherein said method comprises the chloroplast genome transformation of a microalgae with a nucleic acid sequence encoding said recombinant protein, polypeptide or peptide. It further relates to the use of said recombinant peptide, polypeptide or protein for the cosmetic industry.

In recent years, the demand for recombinant protein is increasing more and more because of their high value applications in broad range industries as personal care, cosmetics, healthcare, agriculture, paper industries and materials. Hundred examples of commercial pharmaceutical proteins produced in various recombinant systems have been launched, as for instance, insulin, human growth hormone, erythropoietin and interferon.

Additionally, large quantities of proteins and peptides are needed for these various industrial applications.

The most current industrial expression systems include the bacteria E. coli, the yeast (S. cerevisiae and P. pastoris) and mammalian cell lines. Emerging technologies are insect cell cultures, plants and microalgae.

However, the expression of recombinant peptides and proteins is still limited, as large efforts are required in order to obtain the desired peptides and proteins with a native conformation, in high amounts and high purity. Even a current bacterial system such as E. coli has limitations at expressing recombinant peptides/polypeptides/proteins. Indeed, formation of insoluble aggregates (or inclusion body) arises due to the lack of sophisticated machinery to perform posttranslational modifications, as for instance disulfide bond formation or glycosylations. This results in poor solubility of the protein of interest and/or in the absence of protein activity.

Interest in microalgae as an alternative platform for recombinant protein production has been gaining in recent years.

Recombinant algae offer several advantages over the other recombinant protein production platforms. Microalgae are photosynthetic unicellular microorganisms with low nutriment requirements to grow. They are capable of photoautotrophic, mixotrophic or heterotrophic growth. The cost of protein production in algae is much lower than other production systems in photoauxotrophic growth. Proteins purified from algae, as from plant, should be free from toxins and viral agents that may be present in preparations from bacteria or mammalian cell culture. Indeed, several microalgae species have the GRAS status (Generally Recognized As Safe) granted by the FDA, as for instance for microalgae, Chlorella vulgaris, Chlorella protothecoides S106, Dunaliella bardawil, Chlamydomonas reinhardtii and for cyanobacteria Arthrospira plantesis.

As in transgenic plants, algae have been engineered, to express recombinant genes from both the nuclear and chloroplast genomes.

In addition, recombinant synthesis of peptides and polypeptides composed of repeat units of specific amino acid sequence is difficult because the DNA sequences encoding the peptides or polypeptides are often subject to genetic recombination resulting in genetic instability and leading often to the production of proteins smaller than the native ones.

Recombinant production of relatively small peptides can be also challengeous because they can self-assemble or be subject to proteolytic degradation.

Moreover, in contrast to plant expression system, algae are robust industrial chassis with competitive production costs reachable at industrial scale in reproducible, sterile and well-controlled production conditions within photobioreactors and fermenters or in a single use wave bag. In addition, they can secrete recombinant proteins outside the cell and thus in the culture media, simplifying the subsequent purification steps. Algae have no seasonality and don't used arable land.

The development of chloroplast transformation in algae for the production of proteins of interest is more recent than in plants and requires improvement. In fact, the production yield of recombinant protein is typically between 0.5 and 5% of the total soluble proteins in Chlamydomonas reinhardtii chloroplast, which is still low in comparison to established microbial platform.

In addition, some mammalian proteins are not easily expressed (Rasala et al., 2010).

The invention concerns the KTTKS peptide (SEQ ID No 1), a very well-known peptide in the cosmetic industry marketed in the derivatized form Palmitoyl-KTTKS (Matrixyl®) which has demonstrated wide biological effects in particular on the synthesis of skin extracellular matrix molecules, such as collagen. The inventors of the present invention have surprisingly been able to produce, in the chloroplast of microalgae, recombinant proteins, polypeptides or peptides of KTTKS (SEQ ID No 1) and their derivatives.

Indeed, homologous recombination phenomenon between similar or identical sequences being highly efficiency in the chloroplast genome, transgene with repeat sequences could have been very instable.

By using said method, endogenous disulfide bond formation which is essential for protein, peptide and polypeptide stability and activity, and increased accumulation of the protein, peptide and polypeptide, is allowed.

The present invention thus relates to a recombinant microalgae comprising a nucleic acid sequence encoding:

(a) a recombinant peptide of SEQ ID No 1;

(b) a recombinant peptide, polypeptide or protein consisting in repeated units of SEQ ID No 1; or

(c) a derivative of (a) or (b);

said nucleic acid sequence being located in the chloroplast genome of microalgae.

The present invention also relates to the use of said recombinant algae for producing:

(a) a recombinant peptide of SEQ ID No 1;

(b) a recombinant peptide, polypeptide or protein consisting in repeated units of SEQ ID No 1; or

(c) a derivative of (a) or (b).

The present invention further relates to a method for producing:

(a) a recombinant peptide of SEQ ID No 1;

(b) a recombinant peptide, polypeptide or protein consisting in repeated units of SEQ ID No 1; or

(c) a derivative of (a) or (b);

in the chloroplast of microalgae, wherein said method comprises the chloroplast genome transformation of microalgae with a nucleic acid sequence encoding said recombinant protein, polypeptide or peptide.

In particular, said method comprises:

i) providing a nucleic acid sequence encoding said recombinant protein, polypeptide or peptide;

(ii) introducing the nucleic acid sequence according to (i) into an expression vector which is capable of expressing the nucleic acid sequence in microalgae host cell; and

(iii) transforming the chloroplast genome of microalgae host cell by the expression vector.

In particular, said method further comprises:

(iv) identifying the transformed microalgae host cell;

(v) characterizing the microalgae host cell for the production of the recombinant protein, polypeptide or peptide expressed from said nucleic acid sequence;

(vi) extracting the recombinant protein, polypeptide or peptide; and optionally;

(vii) purifying the recombinant protein, polypeptide or peptide.

More particularly, the method according to the invention allows to increase accumulation and/or stability and/or solubility and/or folding and/or activity of the recombinant peptides, polypeptides or proteins according to the invention in the chloroplast of microalgae.

In particular, the step (vi) can be followed by a step (vi′) to eliminate the algae debris.

Still particularly, said method further comprises a step (vi″) further to step (vi′), in which the polypeptide or protein is cleaved to allow the release of polypeptide or peptide.

Still particularly, said method further comprises a step (vii′) further to step (vii), in which the polypeptide or protein is cleaved to allow the release of peptide units.

Said cleavage can be carried out by any method known by the man skilled in the art such as the use of suitable endoproteinase.

Said recombinant peptides, polypeptides and proteins produced according to the invention can further be formulated for instance in compositions for use in the cosmetic industry. Such composition can form a cosmetic ingredient concentrated in peptide(s) and/or polypeptide(s) and/or protein(s))

In one embodiment, the peptide(s) and/or polypeptide(s) and/or protein(s) produced are purified (step vii) and then mixed with a physiologically acceptable medium to form the cosmetic composition.

In another embodiment, the aqueous solution of peptide(s), polypeptide(s) and/or protein(s) obtained after step (vi′) is used as such or without a complete purification, optionally after being concentrated to a desired content, in combination if necessary with a physiologically acceptable medium, to form the cosmetic composition.

As the peptide(s), polypeptide(s) and/or protein(s) of the invention are produced in an aqueous medium, one important advantage of the invention is the possibility to avoid the use of non-sustainable organic solvents (for example CMR solvents) both for the producing steps and for optional post treatments.

In still another embodiment, the peptide(s)/polypeptide(s)/protein(s) produced and purified for instance by the methods described in the invention, can be chemically modified at their N- or C-terminus.

More particularly, the produced and purified peptide(s)/polypeptide(s)/protein(s) can be chemically modified at the N-terminus by an acyl group (—CO—R₁) or a sulfonyl group (—SO₂—R₁), and/or at the C-terminus by an OR₁, NH₂, NHR₁ or NR₁R₂ group; with R₁ and R₂ being, independently from each other, selected from a biotinyl radical, an alkyl, aryl, aralkyl, alkylaryl, alkoxy or aryloxy radical, that can be linear, branched, cyclic, polycyclic, unsaturated, hydroxylated, carbonylated, phosphorylated and/or sulphured, said radical having 1 to 24 carbon atoms and can contain in its skeleton an O, S and/or N heteroatom.

According to preferred features, R₁ and/or R₂ is an alkyl chain of 1 to 24 carbon atoms, preferably a lipophilic alkyl chain of 3 to 24 carbon atoms ; and/or the peptide or polypeptide or protein is modified by an acyl radical —CO—R₁ at the N-terminus and non-modified at the C-terminus, the acyl radical being preferably selected from octanoyl (C₈), decanoyl (C₁₀), lauroyl (C₁₂), myristoyl (C₁₄), palmitoyl (C₁₆), stearoyl (C₁₈), biotinoyl, elaidoyl, oleoyl and lipoyl; more preferably selected from a lauroyl (C₁₂), myristoyl (C₁₄) and palmitoyl (C₁₆).

The peptide(s)/polypeptide(s)/protein(s) of the invention can also be further prepared and used in a vectorized form, in particular in an encapsulated form, (such as macrocapsules, microcapsules or nanocapsules, macrospheres, microspheres, or nanospheres, liposomes, oleosomes or chylomicrons, macroparticles, microparticles or nanoparticles, macro-sponges, micro-sponges or nanosponges, microemulsions or nanoemulsions, or adsorbed on polymers powdery organic, talcs, bentonites, spores or exines and other mineral or organic carriers).

These compositions comprising the peptide(s) or polypeptide(s) or proteins(s) of the invention when applied to the skin and/or its appendages (hair, body hair, eyelashes, eyebrows, nails, etc.) are able to beautify the skin and its appendages, by improving their general state, comprising for example the following biological effects: strengthening, antioxidant, tensing, hydrating, moisturizing, nourishing, protective, smoothing, reshaping, volumizing (lipofiling), acting on the radiance of the complexion, anti-dark spots, concealer, anti-glycation, anti-aging, anti-wrinkle and fine lines, slimming, soothing, myo-relaxing, lightening, anti-redness, anti-stretch marks, etc.

Therefore, the present invention also provides the use of a peptide and/or polypeptide and/or protein aqueous mixture produced according to the method of the invention and free of algae debris for manufacturing a cosmetic composition. The present invention also provides the use of a peptide and/or polypeptide and/or protein aqueous mixture produced according to the method of the invention and free of algae debris for use in a cosmetic non-therapeutical treatment.

By “recombinant peptide/polypeptide/protein”, is meant in the art, and in the context of the present invention, an exogenous peptide/polypeptide/protein expressed from a recombinant gene (or recombinant nucleic acid sequence) i.e. an exogenous gene (or exogenous nucleic acid sequence) being from a different species (heterologous) or from the same species (homologous).

By “recombinant microalgae” is meant a microalgae comprising a nucleic acid sequence encoding a recombinant protein, polypeptide or peptide. In the context of the invention, the recombinant microalgae is transformed as further detailed below.

By “peptides”, “polypeptides”, “proteins” is meant the meaning commonly understood by a person skilled in the art to which this invention belongs. In particular, peptides, polypeptides and proteins are amino acid polymers linked via peptide (amide) bonds.

More particularly, proteins according to the invention have unique and stable three-dimensional structure and are composed of more than 50 amino acid, like proteins of 54, 60, 66, 72, 75, 78, 84, 90, 96, 100, 102, 108, 114, 120, 150, 180, 200, 300, 350 or more amino acids; peptides according to the invention, also called oligopeptides, are short peptides for examples of 2 to 10 amino acids, like peptides of 3, 4, 5, 6, 7, 8, 9 or 10 amino acids; polypeptides according to classical meaning can be composed of 11 to 50 amino acids, like polypeptides of 11, 12, 15, 18, 20, 24, 25, 30, 35, 36, 40, 42, 45, 48, or 50 amino acids. In the context of the invention, proteins and polypeptides are a repetition of n units of identical or different amino acid sequence, i.e. a repetition of n units of identical or different peptides, n being from 2 to 400, in particular from 2 to 100.

In particular, peptides according to the invention can be the peptide KTTKS of SEQ ID No 1 (named NY2), or its derivatives.

By “derivatives” of peptide of SEQ ID No 1 is meant that the amino acid sequence of the native peptide is mutated or contains one supplementary amino acid or more at its N- or C-terminus. This supplementary amino acids can be any amino acids. In particular, derivatives of the peptide KTTKS of SEQ ID No 1 contain the KTTKS sequence with one or more amino acids at its N- and/or C-terminus.

In particular, the supplementary amino acids in the derivatives of the peptide of SEQ ID No 1 can be a lysine (K), a threonine (T), a serine (S), an aspartic acid (D), a glutamic acid (E), or a glycine (G).

For example, a derivative of the peptide KTTKS of SEQ ID No 1 can be the peptide of SEQ ID No 2 (GKTTKS) (named GNY2).

Other examples are the peptides of SEQ ID No 3 (KTTKSD) (named NY3a), of SEQ ID No 4 (KTTKSE) (named NY3b), of SEQ ID No 5 (DKTTKS) or of SEQ ID No 6 (EKTTKS), and derivatives of GKTTKS that can be peptides of SEQ ID No 7 (GKTTKSD) or SEQ ID No 8 (GKTTKSE).

Polypeptides according to the invention consist, as previously mentioned, in repeated units of SEQ ID No 1, i.e. they contain several fold n repeats of the peptide KTTKS of SEQ ID No 1 or one of its derivatives.

By “derivatives” of said polypeptides according to the invention is meant that the amino acid sequence of the native peptide, which is repeated, is mutated or contains one supplementary amino acid or more at its N- or C-terminus. These supplementary amino acids can be any amino acids.

Examples of such derivatives are named (NY3a)ₙ and (NY3b)ₙ and contain several fold (n) repeats of the peptide NY3a or NY3b previously described.

In particular, such derivatives contain a repetition of five units of NY3a or NY3b and are named respectively (NY3a)×5 and (NY3b)×5.

More particularly, polypeptides derivatives according to the invention are polypeptides of SEQ ID No 9 (KTTKSDKTTKSDKTTKSDKTTKSDKTTKSD) named (NY3a)×5 or of SEQ ID No 10 (GKTTKSDKTTKSDKTTKSDKTTKSDKTTKSD) named G((NY3a)×5) or of SEQ ID No 11 (KTTKSEKTTKSEKTTKSEKTTKSEKTTKSE) named (NY3b)×5 or of SEQ ID No 12 (GKTTKSEKTTKSEKTTKSEKTTKSEKTTKSE) named G((NY3b)×5).

By “mutated” peptide or polypeptide or protein is meant that the nucleic or amino acid sequence of the “mutated” peptide, polypeptide or protein contains one or more mutations in comparison to the nucleic or amino acid sequence of the peptide of SEQ ID No 1 and of the peptides, polypeptides or proteins consisting in repeated units of SEQ ID No 1.

These mutations include deletions, substitutions, insertions and/or cleavage of one or more nucleic acids or amino acids.

According to the invention, a nucleic acid sequence encoding said recombinant protein, polypeptide or peptide is a nucleic acid sequence encoding the proteins, peptides or polypeptides mentioned above.

In particular, nucleic acid sequence encoding peptides and polypeptides of the invention are contemplated in the table 1 below.

TABLE 1

| Name of the | | |
|---|---|---|
| peptide or | | |
| polypeptide | Amino acid sequence | Nucleic acid Sequence |
| NY2 | KTTKS (SEQ ID No 1) | AAAACAACTAAATCA (SEQ ID No 13) |
| GNY2 | GKTTKS (SEQ ID No 2) | GGTAAAACAACTAAATCA (SEQ ID No 14) |
| (NY3a)x5 | KTTKSDKTTKSDKTTKSDKTTKSDKTTKS | AAGACTACCAAAAGTGATAAAACAACTAAAAGCGATAA |
| | D (SEQ ID No 9) | GACAACTAAATCTGATAAAACAACTAAATCAGACAAAAC |
| | | AACAAAATCAGAT (SEQ ID No 15) |
| G(NY3a)x5 | GKTTKSDKTTKSDKTTKSDKTTKSDKTTK | GGTAAGACTACCAAAAGTGATAAAACAACTAAAAGCGA |
| | SD (SEQ ID No 10) | TAAGACAACTAAATCTGATAAAACAACTAAATCAGACAA |
| | | AACAACAAAATCAGAT (SEQ ID No 16) |
| (NY3b)x5 | KTTKSEKTTKSEKTTKSEKTTKSEKTTKSE | AAAACAACTAAATCAGAAAAAACTACAAAAAGTGAGAA |
| | (SEQ ID No 11) | GACTACAAAATCTGAGAAAACAACCAAGTCAGAAAAGA |
| | | CAACAAAATCAGAA (SEQ ID No 17) |
| G(NY3b)x5 | GKTTKSEKTTKSEKTTKSEKTTKSEKTTKS | GGTAAAACAACTAAATCAGAAAAAACTACAAAAAGTGA |
| | E (SEQ ID No 12) | GAAGACTACAAAATCTGAGAAAACAACCAAGTCAGAAA |
| | | AGACAACAAAATCAGAA (SEQ ID No 18) |

In one embodiment, derivatives according to the invention consists in an amino acid sequence at least 80% identical to the amino acid sequence of the recombinant peptide, of SEQ ID No 1 or of the recombinant peptide, polypeptide or protein consisting in repeated units of SEQ ID No 1

By “an amino acid sequence at least 80% identical” is meant in particular, an amino acid sequence 81, 82, 85, 90, 91, 92, 93, 94, 95, 96, 97, 98 or 99% identical. By an amino acid sequence at least 95% “identical” to a query amino acid sequence of the present invention, it is intended that the amino acid sequence of the subject polypeptide is identical to the query sequence except that the subject amino acid sequence may include up to five amino acid alterations per each 100 amino acids of the query amino acid sequence. In other words, to obtain an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS needle (global) program with a “Gap Open” parameter equal to 10.0, a “Gap Extend” parameter equal to 0.5, and a Blosum62 matrix.

Amino acid sequence “at least 80%, 85%, 90%, 95% or 99% identical” to a reference sequence may comprise mutations such as deletions, insertions and/or substitutions compared to the reference sequence. In case of substitutions, amino acid sequence at least 80%, 85%, 90%, 95% or 99% identical to a reference sequence may correspond to a homologous sequence derived from another species than the reference sequence. In another preferred embodiment, the substitution preferably corresponds to a conservative substitution as indicated in the table 2 below.

TABLE 2

| Conservative | |
|---|---|
| substitutions | Type of Amino Acid |
| Ala, Val, Leu, Ile, Met, | Amino acids with aliphatic hydrophobic |
| Pro, Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

Still particularly, nucleic acid sequence encoding derivatives according to the invention consists in a nucleic acid sequence at least 80% identical to the nucleic acid sequence encoding the recombinant peptide of SEQ ID No 1 or of the recombinant peptide, polypeptide or protein consisting in repeated units of SEQ ID No 1.

For example, said nucleic acid sequence is at least 80% identical to any of SEQ ID No 13 to 18.

By “a nucleic acid sequence at least 80% identical” is meant in particular, a nucleic acid sequence 81, 82, 85, 90, 91, 92, 93, 94, 95, 96, 97, 98 or 99% identical. For example, a nucleic acid sequence 95% “identical” to a query sequence of the present invention, is intended to mean that the sequence of the polynucleotide is identical to the query sequence except that the sequence may include up to five nucleotide alterations per each 100 nucleotides of the query sequence. In other words, to obtain a polynucleotide having a sequence at least 95% identical to a query sequence, up to 5% (5 of 100) of the nucleotides of the sequence may be inserted, deleted, or substituted with another nucleotide. In other terms, the sequences should be compared on their entire length (i.e. by preparing a global alignment). For example, a first polynucleotide of 100 nt (nucleotides) that is comprised within a second polynucleotide of 200 nt is 50% identical to said second polynucleotide. The needle program, which uses the Needleman-Wunsch global alignment algorithm (Needleman and Wunsch, 1970, A general method applicable to the search for similarities in the amino acid sequence of two proteins, J. Mol. Biol. 48:443-453) to find the optimum alignment (including gaps) of two sequences when considering their entire length, may for example be used. Preferably, the percentage of identity in accordance with the invention is calculated using the needle program with a “Gap open” parameter equal to 10.0, a “Gap Extend” parameter equal to 0.5, and a Blosum 62 matrix. The needle program is for example available on the ebi.ac.uk World Wide Web site.

In one embodiment, the nucleic acid sequence encoding the protein, polypeptide or peptide according to the invention is codon optimized for expression in the chloroplast genome of the microalgae host cell.

As mentioned above, the nucleic acid sequence according to the invention is introduced into an expression vector which is capable of expressing the nucleic acid sequence.

By “introduction” is meant cloning the nucleic acid sequence encoding the recombinant protein/polypeptide/peptide inside the expression vector with the methods well known by the skilled man and in the way to lead to the expression of this nucleic acid sequence.

“Expression vector” or “transformation vector” or “recombinant DNA construct”, or similar terms, are defined herein as DNA sequences that are required for the transcription of recombinant genes and the translation of their mRNAs in the microalgae algae host cells. “Expression vectors” contains one or more expression cassettes for the recombinant genes (one or more gene encoding the protein, peptide or polypeptide of interest and often selectable markers). In the case of chloroplast genome transformation, expression vectors also contain homologous recombination regions for the integration of expression cassettes inside the chloroplast genome.

In the context of the invention, the expression vector can be in particular a circular molecule with a plasmid backbone containing the two homologous recombination regions and flanking the expressions cassettes, or a linearized molecule corresponding to the expression vector linearized by enzymatic digestion or to a PCR fragment containing only the expression cassettes flanked by the two homologous recombination regions.

In particular, expression vectors of the invention comprise at least one expression cassette and are for example vectors pNY18, pNY19, pNY13, pNY14, pNY15 or pNY16.

“Expression cassette” contains a coding sequence fused operationally to one or more regulatory elements or regulatory sequences, as for instance, fused at its 5′end to a promoter and/or 5′UTR and at its 3′end to a 3′UTR.

The “coding sequence” is the portion of a gene and of its corresponding transcribed mRNA which is translated into the recombinant protein/polypeptide/peptide. The coding sequence includes, for example, a translation initiation control sequence and a stop codon. In some embodiment, the expression cassette can contain a polycistron composed of more than one coding sequence encoding several proteins under the control of only one promoter/5′UTR and 3′UTR.

Said expression cassettes are flanked by left (LHRR) and right (RHRR) endogenous sequences identical to those surrounding the targeted integration site into the chloroplast genome. These left (LHRR) and right (RHRR) homologous regions allow the integration of expression cassettes after homologous recombination exchange between the regions of homology.

Homologous recombination is the ability of complementary DNA sequences to align and exchange regions of homology. Transgenic DNA (“donor”) containing sequences homologous to the genomic sequences being targeted (“template”) is introduced into the organism and then undergoes recombination into the genome at the site of the corresponding genomic homologous sequences.

By its very nature homologous recombination is a precise gene targeting event, hence, most transgenic lines generated with the same targeting sequence will be essentially identical in terms of phenotype, necessitating the screening of far fewer transformation events.

In the case of chloroplast genome transformation of microalgae, the integration of expression cassettes inside the chloroplast genome occurs after homologous recombination between the endogenous homologous sequences of the expression vector with the genome sequences identical or similar to those surrounding the targeted integration site into the chloroplast genome.

In the context of the present invention, other integration sites can be, between the genes rbcL and atpA, or psaB and trnG, or atpB and 16S rDNA, or psaA exon3 and trnE, or trnE and psbH or psbN and psbT, or psbB and trnD.

In some embodiments, in order to enhance its accumulation, the recombinant protein, or polypeptide or peptide can be fused to endogenous proteins, as for instance to the large subunit of ribulose bisphosphate carboxylase (Rubisco LSU). In this case, the promoter and 5′UTR will be those of the endogen rbcL gene, after homologous recombination of the transformation vector into the chloroplast genome.

The protein, peptide or polypeptide of interest will be further separated from RBCL either in vivo or in vitro, depending of the chosen processing system.

In one embodiment, said coding sequence in the expression cassette can also comprise a nucleic acid sequence encoding an epitope tag, in particular the Flag epitope Tag, more particularly the Flag Tag repeat 3 times (3×Flag Tag), in order to identify and/or purify the recombinant protein, polypeptide or peptide.

In particular, said epitope Tag sequence is placed at the N-terminus of the protein, peptide or polypeptide. More particularly, another epitope Tag sequence can be placed at the C-terminus of the protein, peptide, or polypeptide according to the invention, alone or in addition to the one at the N-terminus and this, in order to monitor the release of the peptide/polypeptide/protein of interest to follow its cleavage, for example by an endoprotease.

Examples of epitope Tag sequences are Flag Tag (SEQ ID No 19: DYKDDDDK), 3×Flag Tag (SEQ ID No 20: DYKDDDDKDYKDDDDKDYKDDDDK), HA Tag (SEQ ID No 21: YPYDVPDYA), 3×HA Tag (SEQ ID No 22: YPYDVPDYAYPYDVPDYAYPYDVPDYA), His Tag (SEQ ID No 23. HHHHHH).

“Promoter” as used herein, refers to a nucleic acid control sequence that directs transcription of a nucleic acid.

“5′UTR” or 5′ untranslated region (also known as a leader sequence or leader RNA) is the region of an mRNA that is directly upstream from the initiation codon.

“3′UTR” or 3′ untranslated region is the section of messenger RNA (mRNA) that immediately follows the translation termination codon.

5′UTR and 3′UTR are required for transcript (mRNA) stability and translation initiation.

For microalgae chloroplast expression, promoters, 5′UTRs and 3′UTRs that can be used in the context of the invention are for example: the promoters and 5′UTRs of the genes psbD, psbA, psaA, atpA, and atpB, the 16S rRNA promoter (Prrn) promoter fused with a 5′UTR, the psbA 3′UTR, the atpA 3′UTR or the rbcL 3′UTR.

A 5′UTR from exogenous origin as for instance the 5′UTR of the gene 10L of the bacteriophage T7 can be used also fused downstream a microalgae promoter. In particular, the nucleic acid sequence is operationally linked at its 5′end to the Chlamydomonas reinhardtii 16S rRNA promoter (Prrn).

Stable expression and translation of the nucleic acid sequence according to the present invention can for example be controlled by the promoter and 5′UTR from psbD and the atpA 3′UTR.

Also, in one embodiment of the recombinant microalgae or method according to the invention, nucleic acid sequence encoding a recombinant protein, polypeptide or peptide is operably linked to at least one regulatory sequence chosen from the psbD promoter and 5′UTR (SEQ ID No 78) or the 16S rRNA promoter (Prrn) promoter fused with the atpA 5′UTR (SEQ ID No 76), the psaA promoter and 5′UTR, the atpA promoter and 5′ UTR, the atpA 3′UTRs (SEQ ID No 79) and rbcL 3′UTRs (SEQ ID No 77).

In one embodiment, the promoter less gene encoding the protein, peptide or polypeptide of interest can be integrated after homologous recombination region inside the chloroplast genome just downstream a native promoter.

As mentioned above, the chloroplast genome of microalgae host cell is transformed by the expression vector. This genetic transformation of microalgae host cells, and more particularly the chloroplast genome of microalgae, by expression vector according to the invention can be carried out according to any suitable techniques well known by the man skilled in the art including, without limitations biolistics (Boynton et al., 1988; Goldschmidt-Clermont, 1991), electroporation (Fromm et al., Proc. Natl. Acad. Sci. (USA) (1985) 82:5824-5828; see Maruyama et al. (2004), Biotechnology Techniques 8:821-826), glass bead transformation (Purton et al., revue), protoplasts treated with CaCl₂ and polyethylene glycol (PEG) (see Kim et al. (2002), Mar. Biotechnol. 4:63-73) or microinjection.

In particular, said transformation uses the helium gun bombardment technique of gold micro-projectiles complexed with transforming DNA.

To identify the microalgal transformants, a selectable marker gene may be used. Mention may be made for example of the aadA gene coding aminoglycoside 3″-adenylyltransferase and conferring the resistance to spectinomycin and streptomycin in the case of Chlamydomonas reinhardtii chloroplast transformation. In another embodiment, the selectable marker gene can be the Acinetobacter baumannii aphA-6 Ab gene encoding 3′-aminoglycoside phosphotransferase type VI and conferring the kanamycin resistance.

Chloroplast genome engineering can thus be performed using selectable maker gene conferring resistance to antibiotic or using rescue of photosynthetic mutant.

In particular, in one embodiment, the expression vector for chloroplast genome transformation comprises two expression cassettes comprising the nucleic acid sequence encoding the recombinant protein, polypeptide or peptide according to the invention the selectable marker gene.

More particularly, the expression vector for chloroplast genome transformation comprises one expression cassette comprising the nucleic acid sequence encoding the recombinant protein, polypeptide or peptide according to the invention and one expression cassette comprising the aadA gene coding aminoglycoside 3″-adenylyltransferase (as for instance in FIG. 2).

In another embodiment of the invention, corresponding to the case of rescue of photosynthetic mutant sensitive to light, the expression vector comprises the wild type RHRR region which is deleted in the mutant strain.

After homologous recombinations, the deleted region is restored in the genome of the photosynthetic mutant which is able then to growth under light.

In particular, the coding sequence of the expression cassette according to the invention also comprises a nucleic acid sequence encoding a signal peptide. By “signal peptide” (SP) is meant in the present invention an amino acid sequence placed at the N-terminus of a newly synthetized recombinant protein or polypeptide or peptide. This signal peptide should allow the translocation and the accumulation of the protein inside the lumen of chloroplast thylakoids and not in the chloroplast stroma. The signal peptide is cleaved after translocation across the thylakoid membranes.

For example, such signal peptide sequence is chosen from known protein translocated inside the lumen of thylakoids using either the twin-arginine protein translocation (Tat) pathway or the Sec pathway.

For instance, signal peptides can derive from algae proteins localized in the thylakoid lumen, as the signal peptide from the Chlamydomonas reinhardtii 16 and 23 kDa subunits of the oxygen-evolving complex of photosystem II, or the Chlamydomonas reinhardtii Rieske subunit of b₆f complex or the cryptophytes phycoerythrin alpha subunit (as example from Guillardia theta).

In particular, the Signal Peptide can be extracted from bacterial protein, as the sequence of the E. coli TorA gene encoding the Trimethylamine-N-oxide reductase 1 (UniProt number P33225) (SP; SEQ ID No 24: NNNDLFQASRRRFLAQLGGLTVAGMLGPSLLTPRRATAAQA and nucleic acid sequence of SEQ ID No 80). This signal peptide used the Tat system. This amino acid sequence is cleaved form the protein after the translocation of the later one across the thylakoid membrane.

Other signal peptides can be used which don't leave supplementary amino acid at the N-terminus of the recombinant protein as in particular signal peptide from algae, and in particular from Chlamydomonas reinhardtii.

In one embodiment, the recombinant protein or polypeptide or peptide can be produced as a fusion protein.

The invention thus also relates to a recombinant microalgae or method according to the invention, wherein the nucleic acid sequence encoding a recombinant protein, polypeptide or peptide is fused operationally at its 5′ or 3′end to a nucleic acid sequence encoding a carrier.

Fusion partners or carriers have been developed in recombinant protein production in order to increase accumulation yields, and/or solubility and/or folding and/or to facilitate protein purification. Fusion partners of different sizes (or molecular weight) have been used in various production systems in order to enhance protein solubility and accumulation (maltose-binding protein (MBP), glutathione-S-transferase (GST), thioredoxin, GB1, N-utilizing substance A (NusA), ubiquitin, small ubiquitin-like modifier (SUMO), Fh8) and to facilitate detection and purification (as for examples without limitation MBP, GST and small epitope Tag peptides as c-myc Tag, poly-histidine Tag (His Tag), Flag Tag, HA Tag. Another type of fusion Tags used for purification are stimulus response Tags (or environmentally responsive polypeptides) which allow precipitation of the fusion protein when stimulus as modification of temperature or solution ionic strength are adjusted.

In particular, a carrier according to the present invention can be one of the different Chlamydomonas reinhardtii thioredoxins and in particular the Chlamydomonas reinhardtii thioredoxin m encoded by a nucleic acid sequence derived from the sequence XM_001690262 in GenBank or aprotinin. More particularly, the carrier according to the invention is aprotinin.

The carrier and the recombinant protein, polypeptide or peptide are fused together to form a fusion protein.

By «aprotinin» is meant the basic trypsin inhibitor (BPTI), a small single-chain protein cross-linked by three disulfide bridges which consists of 58 amino acid residues with a molecular mass of 6.5 kDa and an isoelectric point of 10.9.

Said protein is well known by the man skilled in the art and is available commercially. It can for example be produced in recombinant systems such as plants (in cytoplasm by nuclear transformation (Pogue et al., 2010), or in thylakoid lumens by chloroplast transformation (Tissot et al., 2008).

Its formula is C₂₈₄H₄₃₂N₈₄O₇₉S₇ and its molar mass 6511.51 g/mol.

The amino acid sequence for aprotinin from Bos Taurus (bovine) is RPDFC LEPPY TGPCK ARIIR YFYNA KAGLC QTFVY GGCRA KRNNF KSAED CMRTC GGA (SEQ ID No 25 and SEQ ID No 81 for the native nucleic acid sequence of the mature Aprotinin from Bos taurus (GenBank Accession Number X05274)).

In the context of the present invention, the term “aprotinin” also covers chimeric aprotinin and mutated aprotinin.

By “chimeric aprotinin” is meant that aprotinin is connected at its N-terminus and/or at its C-terminus to an epitope Tag peptide(s) and/or signal peptide and/or protease recognition cleavage site.

By “mutated” aprotinin is meant that the nucleic or amino acid sequence of the “mutated” aprotinin contains one or more mutations compared to the nucleic or amino acid sequence of aprotinin or chimeric aprotinin used in particular in the present invention. These mutations include deletions, substitutions, insertions and/or cleavage of one or more nucleic acids or amino acids.

The chimeric aprotinin can be for example the protein called 3F/HA-APRO, comprising aprotinin fused at its N-terminus to a HA epitope Tag or a 3×Flag epitope Tag (3F).

Other examples of chimeric aprotinin can be the protein called HA-SP-3F-FX-APRO (SEQ ID No 26 and 27) comprising aprotinin fused at its N-terminus to an amino acid sequence made of a HA epitope Tag (HA) followed by a signal peptide (SP), a 3×Flag epitope Tag (3F), and a cleavage site for Factor Xa (FX; IEGR), or the chimeric aprotinin called HA-SP-3F-APRO comprising aprotinin fused at its N-terminus to a HA Tag followed by the signal peptide SP and a 3×Flag Tag (3F) or a chimeric aprotinin called HA-SP-APRO comprising aprotinin fused at its N-terminus to a HA Tag followed by the signal peptide SP.

Signal peptide is as previously described.

Other signal peptides can be used which does not leave supplementary amino acids at the N-terminus of the recombinant protein.

If the signal peptide is cleaved after translocation into the lumen of chloroplast thyalkoids (or across the thylakoid membranes), two other chimeric aprotinins can be produced in vivo 3F-APRO (SEQ ID No 28) or 3F-FX-APRO (SEQ ID No 29).

In particular, according to the invention, the fusion partner is used to improve the accumulation and/or the stability of recombinant peptides, polypeptides and proteins.

Still particularly, and as mentioned above, said fusion protein also comprises cleavage sites recognized by specific proteases.

Cleavage sites recognized by specific proteases are well known of the man skilled in the art. They are used to separate the aprotinin from the recombinant protein, polypeptide or peptide of interest, in the case the carrier should be removed if it could interfere with the activity or the structure of said protein, polypeptide or peptide and thus with its uses.

In particular, said cleavage sites are an endoprotease and/or endoproteinase recognition sequence (or protease cleavage site or protease recognition site). More particularly, the sequence of said cleavage sites is placed between the two coding sequences (the one of aprotinin and the one of the recombinant protein, polypeptide or peptide of interest according to the invention).

The cleavage of the fusion protein can be performed either in vivo (in the recombinant host cell before extraction or when apply on the skin for cosmetic peptides) or in vitro after extraction and purification by adding protease.

Non limitative examples of proteases are Factor Xa (FX), Tobacco Edge Virus protease (TEV), enterokinase (EK), SUMO protease, Thrombin, Human Rhinovirus 3C Protease (HRV 3C), endoproteinase Arg-C, endoproteinase Asp-C, endoproteinase Asp-N, endoproteinase Lys-C, endoproteinase Glu-C, proteinase K, IgA-Protease, Trypsin, chymotrypsin and Thermolysin.

Self-cleavage peptides can also be used, as for example the Intein system (Yang et al., 2003), the viral 2A system (Rasala et al., 2012) or the site of the preferredoxin from Chlamydomonas (Muto at al., 2009).

In one embodiment, a linker can be placed between aprotinin and the protease cleavage site. Linkers can be classified into three types: flexible, rigid and cleavable. The usual function of linkers is to fuse the two partners of the fusion protein (e.g. flexible linkers or rigid linkers) or to release them under specific conditions (cleavable linkers) or to provide other functions of the proteins in drug design such as improving of their biological activities or their targeted delivery.

In one embodiment of the present invention, the linker can also make the protease cleaving site more accessible to the enzyme if necessary.

In one embodiment, the flexible linker contains small, non-polar (e.g. Gly) or polar (e.g. Ser or Thr) amino acids. Examples of such linkers are given in Chen et al., 2013.

Flexible linkers according to the invention can be LG (RSGGGGSGGGGSGS; SEQ ID No 30) or LGM (RSGGGGSSGGGGGGSSRS; SEQ ID No 31).

In particular and as an example of the present invention, the peptide NY2 or the polypeptides (NY3a)×5 or (NY3b)×5 can be produced in fusion proteins and can be fused at the C-terminus of the chimeric aprotinin HA-SP-3F-FX-APRO.

This fusion partner can be separated from the polypeptide or peptide of interest by the flexible linker LGM followed by a cleavage site for TEV protease (TV; SEQ ID No 32 ENLYFQG) or enterokinase (EK; SEQ ID No 33 DDDDK).

Therefore, in the present invention, the amino acid sequence and nucleic acid sequence of the different fusion proteins produced in independent algae transformants are for example the protein called HA-SP-3F-FX-APRO-LGM-EK-NY2 (SEQ ID No 34 and 35), HA-SP-3F-FX-APRO-LGM-TV-NY2 (SEQ ID No 36 and 37), HA-SP-3F-FX-APRO-LGM-EK-(NY3a)×5 (SEQ ID No 38 and 39), HA-SP-3F-FX-APRO-LGM-TV-(NY3a)×5 (SEQ ID No 40 and 41), HA-SP-3F-FX-APRO-LGM-EK-(NY3b)×5 (SEQ ID No 42 and 43), HA-SP-3F-FX-APRO-LGM-TV-(NY3b)×5 (SEQ ID No 44 and 45).

When a fusion protein with a carrier is involved, step (vi) of the method according to the invention is a step of purifying the fusion protein.

In that case, the method comprises optionally a step (vii) in which the fusion protein is cleaved.

Said cleavage can be carried out by any method known by the man skilled in the art such as the use of suitable protease to release the recombinant peptide, polypeptide or protein.

Said step (vii) is optionally followed by a purification step (viii) of the recombinant protein, polypeptide or peptide.

In particular, said method further comprises a step (vii') between step (vii) and step (viii), in which the polypeptide is cleaved to allow the release of peptide units.

Said cleavage can be carried out by any method known by the man skilled in the art such as the use of suitable endoproteinase.

Identification of fusion or recombinant proteins can be carried out by Western blot using specific antibodies.

Characterization of the microalgae host cell producing the recombinant protein, polypeptide or peptide can be conducted by techniques known by the man skilled in the art, for example by PCR screening of the antibiotic resistant transformants or Western Blot analysis performed on total protein extracts.

Extraction of total proteins can be carried out using well known techniques (centrifugation, lysis (chemically, mechanically, thermally, enzymatically), sonication, etc).

Elimination of the algae debris can be performed by any adapted method known by the person skilled in the art, such as filtration, precipitation, centrifugation, etc.

Purification can be carried out using well-known techniques. In one embodiment, it comprises an affinity chromatography and/or a step of separation of the peptide, polypeptide or protein according to the invention from the carrier (for example by enterokinase protease digestion) and/or a size exclusion chromatography.

In one embodiment, the step of affinity chromatography can be replaced by an ion exchange chromatography, less expensive for large scale purification.

According to the invention, “microalgae” is a eukarytotic microbial organism that contains a chloroplast or plastid, and optionally that is capable of performing photosynthesis, or a prokaryotic microbial organism (cyanobacteria) capable of performing photosynthesis.

In particular, said microalgae is chosen from the group consisting Chlorophyta (green algae), Rhodophyta (red algae), Stramenopiles (heterokonts), Xanthophyceae (yellow-green algae), Glaucocystophyceae (g laucocystophytes), Chlorarachniophyceae (chlorarachniophytes), Euglenida (eug len ids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae, Phaeophyceae (brown algae).

More particularly, said microalgae is chosen from the group consisting of Chlamydomonas, Chlorella, Dunaliella, Haematococcus, diatoms, Scenedesmaceae, Tetraselmis, Ostreococcus, Porphyridium, and Nannochloropsis.

Even more particularly said microalgae is chosen from the group consisting of Chlamydomonas, more particularly Chlamydomonas reinhardtii, even more particularly Chlamydomonas reinhardtii 137c or a deficient strain as Chlamydomonas reinhardtii CW15.

In particular, said microalgae is cultured in classic conditions known by the man skilled in the art. For example, Chlamydomonas reinhardtii is grown in TAP (Tris Acetate Phosphate) medium to mid-logarithmic phase (densities of approximately 1-2×10⁶ cell/ml), and/or at a temperature comprised between 23° C. to 25° C. (ideally 25° C.), and/or on a rotary shaker in presence of constant light (70-150 μE/m²/s). The experimental part illustrates the conditions of culture.

All the embodiments mentioned in the context of the present invention can be combined.

The above-mentioned recombinant microalgae and method for producing a recombinant protein, polypeptide or peptide in the chloroplast of microalgae can also be applied, at least in part, to the peptides, polypeptides and proteins of:
dipeptides selected from VW, YR, βAH, KT, RT, MO2K, MO₂Ava, AvaMO₂, PR, PA, PM, PP, PG, PS, AP and GP;

These dipeptides are described in WO2001/064178, WO2003/068141, WO1998/007744, WO2010/1336965, FR2668365, WO2006/114657 and WO2010/080376, WO2014/080376.
tripeptides selected from GHK, KAvaK, KFK, KβAK, KAbuK, KAcaK, KPK, KMK, KMOK, KMO₂K, PPS, PPD, PPK, PPL, PPA, PPR, APR, SPR, LPR, QPK, QPH, QPM, QPA, QPR, YPR, LPA, SPA, LPM, IPM, MPL, K(P)HG, K(Pyr)HG, K(Hyp)HG, K(P)GH, K(Pyr)GH, K(Hyp)GH, K(Ac)HG, K(Ac)GH, KHG and KGH;

These tripeptides are described in WO2000/040611, WO2001/043701, WO2000/058347, WO2005/048968, WO2012/164488, WO2012/143845, WO2005/102266, WO2007/093839, WO2010/050191, WO2010/082175, WO2010/082177, WO2015/181688, WO2015/181688, WO2014/080376, WO2017/216177 and WO2016/097966.
tetrapeptides selected from GQPR (SEQ ID No 46), TKPR (SEQ ID No 47), KTFK (SEQ ID No 48), KTAK (SEQ ID No 49), KAYK (SEQ ID No 50), KFYK (SEQ ID No 51), KAvaMO₂K (SEQ ID No 52), KMO2TK (SEQ ID No 53), AQPR (SEQ ID No 54) and AQPK (SEQ ID No 55);

These tetrapeptides are described in WO2000/043417, WO2003/068141, WO2005/048968, WO2012/164488, WO2005/102266, WO2007/093839, WO2019/193113, WO2010/082177 and WO2014/080376.
pentapeptides YGGFL (SEQ ID No 56) or YGGFP (SEQ ID No 57);

These pentapeptides are described in WO2000/015188.
hexapeptides selected from HLDIIW (SEQ ID No 58), HLDIIF (SEQ ID No 59) and HLDIITpi (SEQ ID No 60).

These hexapeptides are described in WO2007/068998, FR2735687, and WO2007/129270.

In these sequences:

MO means a sulfone methionine;

MO2 means a sulfoxide methionine;

Ava corresponds to the 5 amino valeric acid;

Abu corresponds to the amino butyric acid;

K(P) corresponds to a proline grafted on the lateral chain of the lysine amino acid;

K(Pyr) corresponds to a proline grafted on the lateral chain of the lysine amino acid;

K(Hyp) corresponds to a proline grafted on the lateral chain of the lysine amino acid;

K(Ac) corresponds to a proline grafted on the lateral chain of the lysine amino acid; and

Tpi corresponds to the tryptoline-3-carboxylic acid.

These kind of modifications of natural amino-acids can be performed by chemical synthesis after the production of the peptides sequences in the chloroplast of the recombinant microalgae.

A particularly interesting list is the list comprising VW, YR, KT, PA, PM, PP, GHK, KAvaK, KFK, KMO₂K, PPL, PPA, SPR, LPA, SPA, K(P)HG, K(Pyr)HG, K(Hyp)HG, K(P)GH, K(Pyr)GH, K(Hyp)GH, K(Ac)HG, K(Ac)GH, KHG, KGH, GQPR (SEQ ID No 46) and KTFK (SEQ ID No 48).

The content of the above-mentioned patent applications is incorporated by reference.

Said peptides, polypeptides and proteins can further be subject to treatment(s) to obtain compounds formulated for cosmetic industry. They thus can be used in cosmetic compositions.

The above-mentioned recombinant microalgae and method for producing a recombinant protein, polypeptide or peptide in the chloroplast of microalgae can also be applied to polypeptides or proteins designed to further contain peptides (amino acids units) of different biological and physical properties, as for instance adding collagen or silk peptides, or the domain for the fixation of biotin or hyaluronic acid, or heparin binding domains, or growth factors, or protease degradation sites or cell binding domains (as for instance the RGD domain involved in the reconnaissance of fibrillins). Particularly interesting combinations of peptide sequences are combinations of peptides either of same activity to enhance synergistically this activity or peptides of different activities to advantageously provide multiple activities.

The invention will be further illustrated by the following figures and examples.

### FIGURES

FIG. 1: Codon usage in the Chlamydomonas reinhardtii chloroplast genome

FIG. 2: Schematic presentation of the chloroplast transformation vectors for the production of fusion proteins with peptides and polypeptides of KTTKS and their derivatives.

FIG. 3: Western blot analysis of independent algae transformants 137c- or CW-NY18 (A, B) and 137c- or CW-NY19 (C, D) expressing the genes encoding the fusion proteins containing the peptide, using monoclonal anti-Flag M2 antibody. 100 μg or 50 μg of each total soluble protein samples extracted with SDS buffer lysis from NY18 or NY19, respectively, were separated on a 15% SDS polyacrylamide gel. MW: molecular weight standard. 50 μg of total soluble protein samples extracted by sonication from CW-AU76-1 transformant was loaded as positive control (C, D). 100 μg (A, B) or 50 μg (C, D) of each total soluble protein samples extracted with SDS buffer lysis from Wild-type (WT) 137c or CW15 was loaded as negative control. Arrows indicate the positions of recombinant proteins.

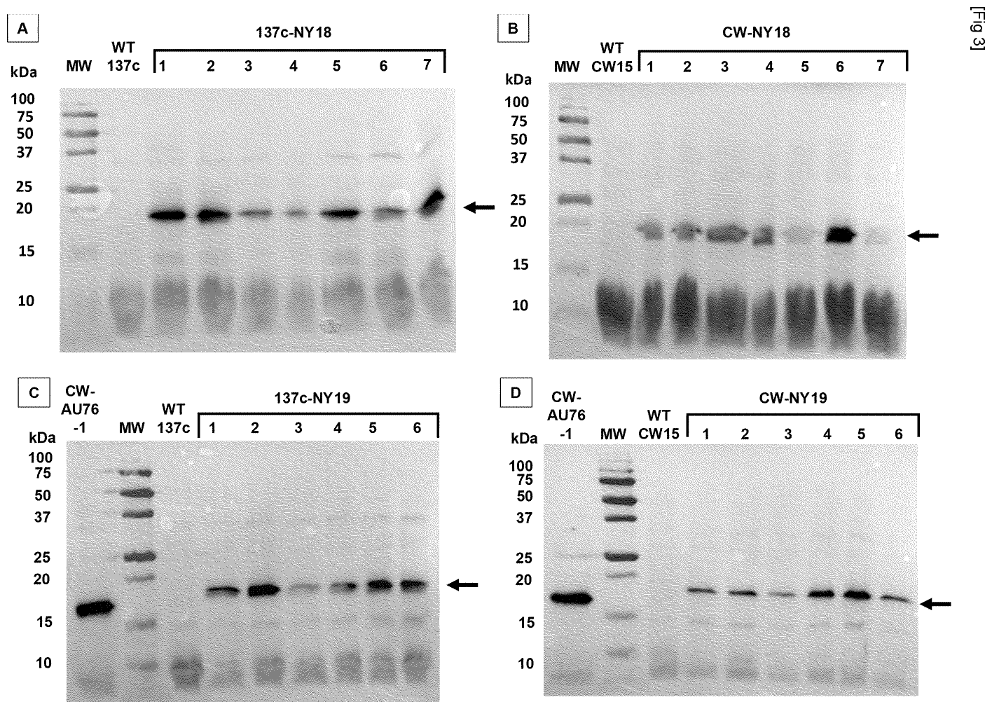

FIG. 4: Western blot analysis of independent algae transformants 137c- or CW-NY13 (A, B) and 137c- or CW-NY14 (C, D) expressing the genes encoding the fusion proteins containing the polypeptide (NY3b)×5, using monoclonal anti-Flag M2 antibody. 100 μg (or 50 μg for NY14 transformants) of each total soluble protein samples extracted with SDS buffer lysis from NY13, respectively, were separated on a 15% SDS polyacrylamide gel. MW: molecular weight standard. 50 μg of total soluble protein samples extracted by sonication from CW-AU76-1 transformant was loaded as positive control. 100 μg (A, B) or 50 μg (C, D) of each total soluble protein samples extracted with SDS buffer lysis from Wild-type (WT) 137c or CW15 was loaded as negative control. Arrows indicate the positions of recombinant proteins.

FIG. 5: Western blot analysis of independent algae transformants 137c- or CW-NY15 (E,F) expressing the genes encoding the fusion proteins containing the polypeptide (NY3a)×5, using monoclonal anti-Flag M2 antibody. 100 μg of each total soluble protein samples extracted with SDS buffer lysis from NY15 and from Wild-type (WT) 137c or CW15 were separated on a 15% SDS polyacrylamide gel. MW: molecular weight standard. 50 μg of total soluble protein samples extracted by sonication from CW-AU76-1 transformant was loaded as positive control. Arrows indicate the positions of recombinant proteins.

FIG. 6: Western Blot analysis of different elution fractions from anti-Flag M2 affinity chromatography performed on a protein extract from CW-NY13-4 (A) and CW-NY18-6 (B) transformants using monoclonal anti-Flag M2 antibody. Different quantities (25 or 50 μg) of protein samples extracted by sonication and/or precipitated by ammonium sulfate or volume of elution fraction were loaded on a 15% SDS polyacrylamide gel. A) CW-NY13-4 SA: precipitated proteins by ammonium sulfate from CW-NY13-4. MW: molecular weight standard. Load: total soluble protein extracted by sonication before the incubation with anti-Flag M2 resin. FT: Flow through. EA: elution fraction. W: wash fraction. Arrows indicate the positions of purified recombinant proteins.

### EXAMPLES

### Example 1

Material and Methods

All oligonucleotides and synthetic genes were purchased from Eurofins. All enzymes were purchased from NEB, Promega, Invitrogen and Sigma Aldrich/Merck. All plasmids were built on the pBluescript II backbone.

### Algal Strains and Growth Conditions

The two algal strains used are the Chlamydomonas reinhardtii wild type (137c; mt+) and the cell wall deficient strain CW15 (CC-400; mt+), obtained from the Chlamydomonas Resource Center, University of Minnesota).

Prior to transformation, all strains were grown in TAP (Tris Acetate Phosphate) medium to mid-logarithmic phase (densities of approximately 1-2×10⁶ cell/mL) at a temperature comprise between 23° C. to 25° C. (ideally 25° C.) on a rotary shaker in presence of constant light (70-150 μE/m²/s).

Transformants were grown in the same conditions and the same media containing 100 μg/mL of spectinomycin or 100 μg/mL kanamycin, depending of the selectable marker gene present in the transformation vector.

Growth kinetics was also followed by measuring the optical density at 750 nm using a spectrophotometer.

### Algal Transformation

Chlamydomonas reinhardtii cells are transformed using the helium gun bombardment technique of gold micro-projectiles complexed with transforming DNA, as described in the article Boynton et al., 1988. Briefly, the Chlamydomonas reinhardtii cells were cultivated in TAP medium until midlog phase, harvested by gentle centrifugation, and then resuspended in TAP medium to a final concentration of 1.10⁸ cells/mL. 300 μL of this cell suspension was plated onto a TAP agar medium supplemented with 100 μg/mL of spectinomycin or 100 μg/mL of kanamycin, depending of the selectable marker gene present in the transformation vector. The plates were bombarded with gold particles (S550d; Seashell Technology) coated with transformation vector, as described by the manufacturer. The plates were then placed at 25° C. under standard light conditions to allow selection and formation of transformed colonies.

### Total DNA Extraction and PCR Screening of Positive Transformants

Total DNA extraction was performed using the chelating resin Chelex 100 (Biorad) from single colonies (with size of around 1 mm in diameter) of wild type and/or antibiotic resistant transformants Chlamydomonas strains.

From isolated colonies, a quantity of cells corresponding to about 0.5 mm in diameter was removed with a pick and resuspended in 20 μL of H₂O. 200 μL of ethanol were added and incubated 1 min at room temperature. 200 μL of 5% Chelex were incorporated and vortexed. After an incubation of 8 min at 100° C., the mixture was cooled down and centrifuged 5 min at 13,000 rpm. Finally, the supernatant was collected.

After transformation, algae colonies growing onto restrictive solid medium plates were expected to have the antibiotic resistant gene and the other transgene(s) incorporated into their genome.

In order to identify stable integration of the recombinant genes into the algal genome, the antibiotic resistant transformants were screened by Polymerase Chain Reaction (PCR or PCR amplification) in a thermocycler using 1 μL of total DNA previously extracted as template, two synthetic and specific oligonucleotides (primers) and Taq polymerase (GoTaq, Promega). The cycles of PCR amplification followed the guidelines recommended by the manufacturer. The PCR reactions were subjected to gel electrophoresis in order to check the PCR fragment of interest.

### Protein Extraction, Western Blot Analyses

Chlamydomonas cells (50 mL, 1-2.10⁸ cells/mL) were collected by centrifugation. Cell pellet was resuspended in lysis buffer (50 mM Tris-HCl pH 6.8, 2% SDS and 10 mM EDTA). In some embodiments of the example, the lysis buffer didn't contain 10 mM EDTA. After 30 min at room temperature, cell debris were removed by centrifugation at 13000 rpm and the supernatant containing the total soluble proteins was collected.

Depending on the further analysis step, total soluble proteins were extracted under non denaturing conditions. Cell pellet was resuspended in a buffer containing 20 mM Tris-HCl pH 6.8. The sonication step was carried out with the algal cell suspension held on ice, using a cell disruptor sonicator FB505 500W (Sonic/FisherBrand) and a setting of the micro-tip probe to 20% power, with continuous sonication for 5 min. After sonication, cell debris were removed by centrifugation at 13000 rpm, 30 min.

Total soluble proteins present in the supernatant were quantified using the Pierce BCA protein assay kit, following the instructions of the supplier (Thermofisher).

Total soluble protein samples (50 or 100 μg or another quantity further mentioned in the example depending of the experiment) were separated in a 12 or 15% Tris-glycine SDS-PAGE prepared according to Laemmli (1970).

For experiments performed under reducing conditions, samples were prepared in Laemmli sample loading buffer with 50 mM DTT (or more depending of the fusion protein) or 5% Beta-mercaptoethanol, and further denaturated 5 min at 95° C. before loading. The SDS PAGE experiments were carried out using a Protein Gel tank from BioRad.

After separation, samples were blotted onto a nitrocellulose membrane (GE HealthCare) using standard transfer buffer and a Trans-Blot® Turbo™ Transfer System from Biorad. In order to visualize the transferred proteins, the nitrocellulose membrane were stained by Ponceau S dye. Membranes were further blocked with Tris-buffered saline Tween buffer (TBS-T) (50 mM Tris-HCl pH 7.5, 150 mM NaCl, 0.1% Tween-20) containing 5% Bovin Serum Albumin (BSA). After one hour of saturation at room temperature under gently shaking, membranes were incubated during one night at 4° C. with TTBS buffer containing mouse primary antibody (See Table 3).

The antibody mentioned in Table 3 below were used as primary antibodies.

TABLE 3
Primary antibodies

| Primary antibody | Source | Dilution |
|---|---|---|
| Monoclonal ANTI-FLAG ® M2 | Sigma | 1:1000 |
| antibody produced in mouse | | |
| Monoclonal ANTI-HA PURIFIED | Sigma | 1:6000 |
| antiobody produced in mouse IGG | | |
| Monoclonal ANTI-Aprotinin | Abcam | 1:2000 |
| antibody produced in mouse | | or |
| | | 1:3000 |

After three washes with TBS-T-BSA buffer, membranes were incubated one hour at room temperature with TBS-T-BSA buffer containing secondary antibodies (Anti-Mouse IgG (H+L), HRP Conjugate; Promega). After four washes with TTBS buffer and one wash with TBS buffer, the membranes were incubated in an enhanced chemiluminescence (ECL) substrate (Clarity Max ECL substrate; Biorad). The ECL signals were visualized with the ChemiDoc™ XRS+ system (Biorad).

### Protein Purification

Depending on the protein and on the further steps to which the protein is submitted, the first or second method below is conducted.

1) After centrifugation, algae cell pellets were resuspended in a buffer containing 50 mM Tris-HCl pH8, 500 mM NaCl and 0.1% Tween 20. Approximately, 10 mL of buffer were used per g of wet algal cells, depending of the transformants. The resuspended cells were sonicated in the same conditions as previously described.

2) After centrifugation, algae cell pellets were resuspended in a buffer containing 20 mM Tris-HCl pH 6.8. The resuspended cells were sonicated in the same conditions as previously described. The total soluble proteins were precipitated with 80% ammonium sulfate as described by Wingfield, 2001. After a 15000 g centrifugation for 30 min at 4° C., the protein pellets were resolubilized in 60 mM Tris-HCl pH8 buffer. These suspensions were dialysed in Slide-A-Lyzer Dialysis Cassettes (3.5 kDa MWCO, Thermo Scientific) as described by the manufacturer against the previous buffer. The next step being an anti-FLAG M2 affinity chromatography, NaCl and Tween 20 were added to the dialyzed samples to adjust the buffer composition to that of the binding buffer hereinafter described.

### Affinity Chromatography

All recombinant proteins were tagged in their N-terminal with a Flag-Tag epitope which will bind specifically on an anti-FLAG M2 affinity gel (Sigma/Merck). This resin contains a mouse monoclonal ANTI-FLAG® M2 antibody that is covalently attached to agarose.

All steps of this experiment were carried out as described by the manufacturer. Briefly, the samples of total soluble proteins were filtered using a cellulose acetate 0.45 μm filter and mixed with anti-FLAG M2 affinity gel prepared as recommended by the manufacturer and equilibrated in binding buffer (50 mM Tris-HCl pH8, 500 mM NaCl, 0.1% Tween 20). Approximately, 1 mL of resin was used per 4 to 8 g of wet algal cells, depending of the transformants. Binding of the recombinant fusion protein was performed at 4° C. for 4 h or overnight with a gently and continuous end-over-end mixing. After incubation, the mixture of soluble protein incubated with resin were loaded by gravity on an empty Bio-rad Econo-pac column or collected by centrifugation, and washed several times with 40 column volumes TBST and 20 column volumes TBS. The protein of interest was eluted from the resin using 100 mM Glycine pH 3.5, 500 mM NaCl and neutralized with Tris-HCl pH 8 to a final concentration of 50 mM. Each elution fraction was further analyzed by SDS-PAGE and Western Blot.

The elution fractions containing the protein of interest were dialyzed in Slide-A-Lyzer Dialysis Cassettes (3.5 kDa MWCO, Thermo Scientific) as described by the manufacturer against the buffer used in the further step, as for instance, for the protease digestion. The dialyzed samples were concentrated using Vivaspin 6 (3 kDa MWCO, GE Healthcare).

Separation of the Protein of Interest from the Carrier

The separation of the protein of interest from the carrier was made by protease digestion, in particular, in the present invention by enterokinase (light chain) or Tobacco Etch Virus (TEV) Protease from New England BioLabs (NEB).

Enzymatic digestions were performed as recommended by the manufacturer.

For example, for enterokinase light chain digestion, reactions combined 25 μg of protein of interest in 20 μL of buffer (20 mM Tris-HCl pH 8.0, 50 mM NaCl, 2 mM CaCl₂), with 1 μL of enterokinase light chain. Incubation was made at 25° C. for 16 h.

For example, for TEV digestion, typical reaction recommended by the manufacturer combined 15 μg of protein substrate with 5 μL of TEV protease reaction buffer (10×) to make a 50 μL total reaction volume. After addition of 1 μL of TEV Protease, reaction was incubated at 30° C. for 1 hour or 4° C. overnight.

For example, for Factor Xa digestion, the manufacturer recommended to digest 50 μg of fusion protein with 1 μg of FXa in a volume of 50 μL at 23° C. for 6 h. The reaction buffer consisted in 20 mM Tris-HCl pH 8.0, 100 mM NaCl and 2 mM CaCl₂.

### Cleavage of the Polypeptide by Endoproteinases

The choice of the endoproteinase used to cleave the polypeptide of interest depends of the amino acid sequence of this polypeptide. Endoproteinases can be for instance, endoproteinase Glu-C, endoproteinase Arg-C, endoproteinase Asp-C, endoproteinase Asp-N, or endoproteinase Lys-C.

Enzymatic digestions were performed as recommended by the manufacturer. For example, for endoproteinase Glu-C digestion (from NEB), the manufacturer recommended to digest 1 μg of substrate protein with 50 ng of endoproteinase Glu-C at 37° C. for 16 h. The reaction buffer consisted in 50 mM Tris-HCl pH 8.0 and 0.5 mM GluC-GluC.

### Size Exclusion Chromatography (SEC)

Size-exclusion chromatography of purified and digested fusion protein was performed using an AKTA Pure system (GE Healthcare) in order to separate the protein of interest from the carrier.

A Superdex S30 Increase G10/300 GL column (GE Healthcare) and a HiLoad 26/600 Superdex 30 prep grade column were first calibrated using two standards diluted with 2× PBS buffer (or appropriate buffer for the further step): aprotinin (bovine lung; 6.5 kDa), and glycine (75 Da).

After a washing step in water, the Superdex S30 Increase G10/300 GL column was equilibrated in running buffer (2× PBS, pH 7.4, or appropriate buffer for the further step) and 200 to 500 μL samples were run through the column at a rate of 0.5 mL/min. Elution of protein was detected by measuring optical absorbance at 280, 224 and 214 nm. 0.5 mL fractions were collected and analyzed by SDS-PAGE followed by Western-Blot or stained by Coomassie Blue dye.

After a washing step in water, the HiLoad 26/600 Superdex 30 prep grade column was equilibrated in running buffer (2× PBS, pH 7.4, or the appropriate buffer for the further step) and samples (4 to 30 mL) were run through the column at a rate of 2.6 mL/min. Elution of proteins was detected by measuring optical absorbance at 280, 224 and 214 nm. 4 mL fractions were collected and analyzed by SDS-PAGE followed by Western-Blot.

In some embodiment, the elution fractions of interest were pooled and evaporated using a SpeedVac (Eppendorf). The peptides or polypeptides or proteins present in these evaporated samples were subjected to Edman degradation to confirm the amino acid sequence at the N-terminus of the protein of interest.

### Example 2

Production of Mono and Polypeptides of KTTKS or Derivatives in a Fusion Protein Using Aprotinin as Carrier, in the Chloroplast of Chlamydomonas Reinhardtii by Chloroplast Genome Transformation

Construction of Transformation Vectors (pNY18, pNY19, pNY13, pNY14, pNY15, pNY16)

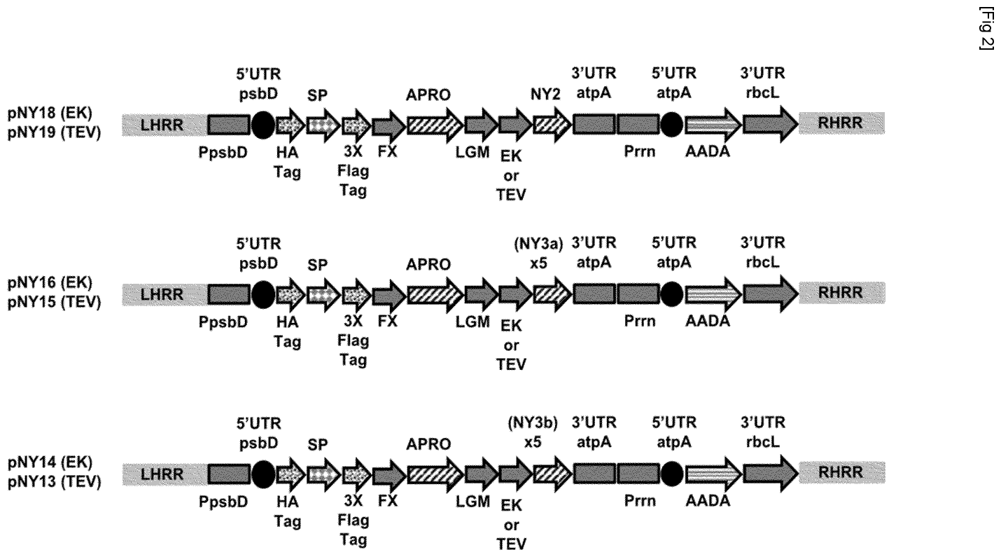

Several chloroplast transformation vectors were constructed in order to express the peptides KTTKS (named NY2) and GKTTKS (named GNY2) and polypeptides of KTTKS derivatives in a fusion protein using aprotinin as a carrier (FIG. 2).

In chloroplast transformation vector, the peptide NY2 and the polypeptides (NY3a)×5 (KTTKSDKTTKSDKTTKSDKTTKSDKTTKSD) (SEQ ID No 9) and (NY3b)×5 (KTTKSEKTTKSEKTTKSEKTTKSEKTTKSE) (SEQ ID No 11) were produced in fusion proteins in which they were fused, as examplified in the present invention, at the C-terminus of the chimeric aprotinin HA-SP-3F-FX-APRO. This fusion partner contained aprotinin fused at their N-terminus to an amino acid sequence made of the HA epitope Tag (HA) followed by the signal peptide (SP), the 3×Flag epitope Tag (3F), and the cleavage site for Factor Xa (FX; IEGR (SEQ ID No 61)).

In the fusion protein, the peptide NY2 or the polypeptides (NY3a)×5 and (NY3b)×5 were separated from the carrier by the flexible linker LGM (RSGGGGSSGGGGGGSSRS) followed by a cleavage site for TEV protease (TV; SEQ ID No 32; ENLYFQG) or enterokinase (EK; SEQ ID No 33; DDDDK).

Therefore, different fusion proteins were produced in independent algae transformants, as for instance, the protein called HA-SP-3F-FX-APRO-LGM-EK-NY2 (SEQ ID No 33 and 34), HA-SP-3F-FX-APRO-LGM-TV-NY2 (SEQ ID No 35 and 36), HA-SP-3F-FX-APRO-LGM-EK-(NY3a)×5 (SEQ ID No 37 and 38), HA-SP-3F-FX-APRO-LGM-TV-(NY3a)×5 (SEQ ID No 39 and 40), HA-SP-3F-FX-APRO-LGM-EK-(NY3b)×5 (SEQ ID No 41 and 42), HA-SP-3F-FX-APRO-LGM-TV-(NY3b)×5 (SEQ ID No 43 and 44) (FIG. 2).

After their production in algae chloroplasts, the signal peptide (SP) will target these fusion proteins into the thylakoids. During protein translocation, the N-terminus fragment HA-SP will be cleaved and the following other recombinant proteins will be produced in vivo, 3F-FX-APRO-LGM-TV-(NY3b)×5 (SEQ ID No 62), 3F-FX-APRO-LGM-EK-(NY3b)×5 (SEQ ID No 63), 3F-FX-APRO-LGM-TV-(NY3a)×5 (SEQ ID No 64), 3F-FX-APRO-LGM-EK-(NY3a)×5 (SEQ ID No 65), 3F-FX-APRO-LGM-EK-NY2 (SEQ ID No 66) and 3F-FX-APRO-LGM-TV-NY2 (SEQ ID No 67).

The release of the peptides and the polypeptides from the chimeric aprotinin will be performed in vitro by site specific proteolysis of the fusion protein with enterokinase or TEV proteases.

After the cleavage of the fusion protein HA-SP-3F-FX-APRO-LGM-TV-NY2 or 3F-FX-APRO-LGM-TV-NY2 by the TEV protease, the released peptide will be GNY2 (GKTTKS). In the case of the TEV digestion of the fusion proteins HA-SP-3F-FX-APRO-LGM-TV-(NY3a)×5 (or 3F-FX-APRO-LGM-TV-(NY3a)×5) and HA-SP-3F-FX-APRO-LGM-TV-(NY3b)×5 (or 3F-FX-APRO-LGM-TV-(NY3b)×5), the released polypeptides will be G((NY3a)×5) and G((NY3b)×5), respectively.

In Chlamydomonas reinhardtii, the codon usage in the nucleic acid sequence encoding protein of interest has been shown to play a significant role in protein accumulation (Franklin et al., 2002; Mayfield and Schultz, 2004).

The nucleic acid sequence encoding the chimeric aprotinin were designed and optimized in order to improve their expression in C. reinhardtii host cells

Methods for altering polynucleotides for improved expression in host cell are known in the art, particularly in algae cell, particularly in C. reinhardtii.

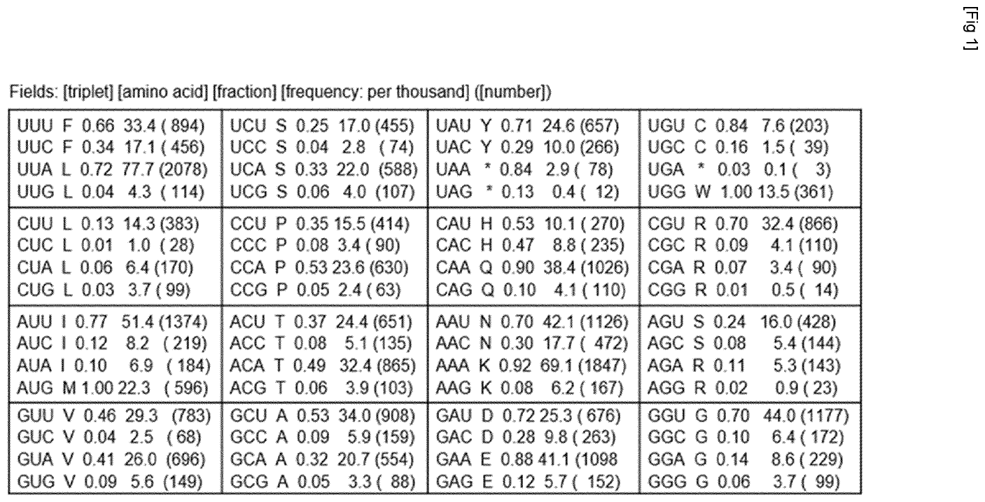

A codon usage database is found at http://www.kazusa.or.jp/codon/. (See the codon usage for chloroplast genome of C. reinhardtii; FIG. 1).

For improving expression in C. reinhardtii chloroplast of the gene of interest in the present invention, codons from their native sequence which are not commonly used, were replaced with a codon coding for the same or a similar amino acid residue that is more commonly used in the codon bias from the C. reinhardtii chloroplast genome. In addition, other codons were replaced to avoid sequences of multiple or extended codon repeats, or some restriction enzyme sites, or having a higher probability of secondary structure that could reduce or interfere with expression efficiency.

In order to check and to fulfill all criteria mentioned above, the amino acid sequence of the protein of interest were also optimized by the software GENEius of Eurofins using the appropriate usage codon for C. reinhardtii chloroplast genome.

After optimization, the gene encoding aprotinin (APRO) were operationally fused at its 5′end to a codon optimized nucleic acid sequence encoding the HA epitope Tag (HA) followed by a signal peptide, the 3×Flag epitope Tag (3F) and the cleavage site recognized by the Factor Xa protease (FX) to form the chimeric aprotinin HA-SP-3F-FX-APRO (SEQ ID No 26 AND 27).

The nucleic acid sequence encoding the recombinant peptide of KTTKS or GKTTKS, or polypeptide of KTTKS or their derivatives were designed and optimized as mentioned above in order to improve their expression in C. reinhardtii host cells.

After codon optimization, the different synthetic genes GNC-LENY3a2 (SEQ ID No 68), GNC-LENY3b1 (SEQ ID No 69), GNC-LTNY3a2 (SEQ ID No 70) and GNC-LTNY3b1 (SEQ ID No 71) encoding respectively the polypeptides (NY3a)×5, (NY3b)×5), G((NY3a)×5), G((NY3b)×5) were synthetized by Eurofins. These optimized genes were cloned by the Gibson assembly method downstream the gene encoding the carrier into an expression cassette present in the chloroplast transformation vector pAU76 linearized by PmeI to give respectively, pNY16, pNY14, pNY15, and pNY13.

The chloroplast transformation vectors pNY13 and pNY14 allowed the expression of the polypeptide (NY3b)×5 in the fusion proteins HA-SP-3F-FX-APRO-LGM-TV-(NY3b)×5 and HA-SP-3F-FX-APRO-LGM-EK-(NY3b)×5, respectively (FIG. 2).

The chloroplast transformation vectors pNY15 and pNY16 allowed the expression of the polypeptide (NY3a)×5 in the fusion proteins HA-SP-3F-FX-APRO-LGM-TV-(NY3a)×5 and HA-SP-3F-FX-APRO-LGM-EK-(NY3a)×5, respectively (FIG. 2).

The optimized genes GNC-ALENY2 (SEQ ID No 72) and GNC-ALTNY2 (SEQ ID No 73) were cloned by Gibson assembly method into an expression cassette present in the chloroplast transformation vector pLE63 linearized by BamHI and PmeI digestions to give respectively, pNY18 and pNY19.

The chloroplast transformation vectors pNY18 and pNY19 allowed the expression of the peptide NY2 in the fusion proteins HA-SP-3F-FX-APRO-LGM-EK-NY2 and HA-SP-3F-FX-APRO-LGM-TV-NY2, respectively (FIG. 2).

The expression vectors pAU76 and pLE63 for chloroplast genome transformation contained two expression cassettes (FIG. 2) for the expression of the genes encoding the selectable marker and the fusion protein.

These two expression cassettes are flanked by a left (LHRR) and right (RHRR) endogenous homologous recombination sequences which are identical to those surrounding the targeted integration site into the C. reinhardtii chloroplast genome. In a preferred embodiment, the chloroplast transformation vectors in the present invention allow the targeted integration of the transgenes into the chloroplast genome of C. reinhardtii between the 5S rRNA and psbA genes (and derives from instance from GenBank Accession Number NC005352).

The selectable marker gene was the aadA gene coding aminoglycoside 3″-adenylyltransferase and conferring the resistance to spectinomycin and streptomycin. The gene is operationally linked at its 5′ end to the C. reinhardtii 16S rRNA promoter (Prrn) fused to the atpA 5′UTR (SEQ ID No 76) and at its 3′ end to the 3′UTR of the C. reinhardtii rbcL gene (SEQ ID No 77) (FIG. 2).

Stable expression and translation of the fusion protein gene were controlled by the promoter and 5′UTR from the C. reinhardtii psbD (SEQ ID No 78) and the 3′UTR from C. reinhardtii atpA (SEQ ID No 79) (FIG. 2).

Transformation of Algae

The transformation vectors pNY18, pNY19, pNY13, pNY14, pNY15, pNY16 were bombarded in C. reinhardtii cell (137c and CW15) as described in the Example 1. In order to identify stable integration of the recombinant genes encoding fusion protein into the chloroplast algal genome, spectinomycin resistant colonies were screened by PCR analysis. For positive PCR screens of the fusion protein gene, the primers O5′ASTatpA2 5′-CCTACTTAATTAAAAACTGCAGTAGCTAGCTCTGC-3′ (SEQ ID No 74) and O3′SUTRpsbD 5′-CGATGAGTTGTTTTTTTATTTTGGAGATACACGC-3′ (SEQ ID No 75) annealing, respectively, in the atpA 3′UTR and psbD 5′UTR were used.

Analyses and Results

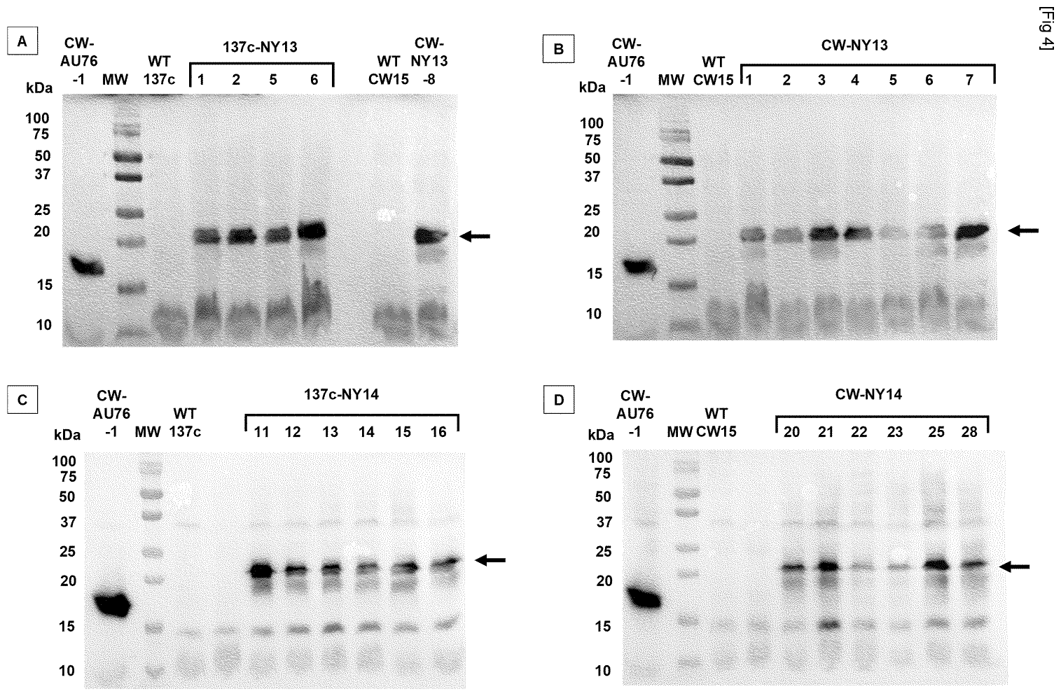

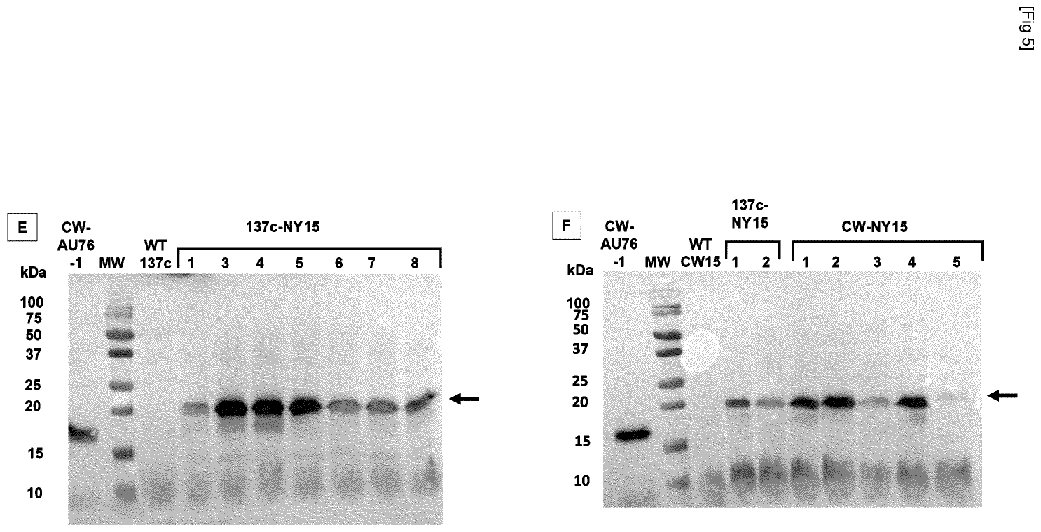

Western Blot analysis of total soluble proteins extracted from different independent strains transformed with different expression vectors revealed that the fusion proteins HA-SP-3F-FX-APRO-LGM-EK-NY2, HA-SP-3F-FX-APRO-LGM-TV-NY2, HA-SP-3F-FX-APRO-LGM-EK-(NY3a)×5, HA-SP-3F-FX-APRO-LGM-TV-(NY3a)×5, HA-SP-3F-FX-APRO-LGM-EK-(NY3b)×5, HA-SP-3F-FX-APRO-LGM-TV-(NY3b)×5 were well produced (FIGS. 4 and 5).

Moreover, in all transformants, the HA epitope Tag and the signal peptide seems to be cleaved because Western blots performed on the same total soluble protein extracts showed that the primary anti-HA antibody didn't recognize any fusion protein. Thus, the fusion proteins produced in the different transformants would be 3F-FX-APRO-LGM-EK-NY2, 3F-FX-APRO-LGM-TV-NY2, 3F-FX-APRO-LGM-EK-(NY3a)×5, 3F-FX-APRO-LGM-TV-(NY3a)×5, and 3F-FX-APRO-LGM-EK-(NY3b)×5.

The comparison of the fusion protein amounts between the different types of transformants, performed by Western blot analyses, showed that among all producing transformants, the clones CW-NY13-4 and CW-NY18-6 produced high levels of 3F-FX-APRO-LGM-TV-(NY3b)×5 (0.1% of total soluble proteins; TSP) and 3F-FX-APRO-LGM-EK-NY2 (0.089% TSP), respectively. Thus, these two transformants were used for larger scale production.

### Example 3

Purification of Peptides and Polypeptides of KTTKS or Derivatives

About 80 g of cells for each transformants CW-NY13-4 and CW-NY18-6 were produced from several 1 L cascade cultures. Algae cells were resuspended and sonicated as described in the Material and Methods. 850 mL of total soluble protein extract for each transformants CW-NY13-4 and CW-NY18-6 were obtained.

In order to concentrate these extract volumes of 850 mL, a supplementary steps of ammonium sulfate precipitation and dialysis were added before the affinity chromatography as described in example 1.

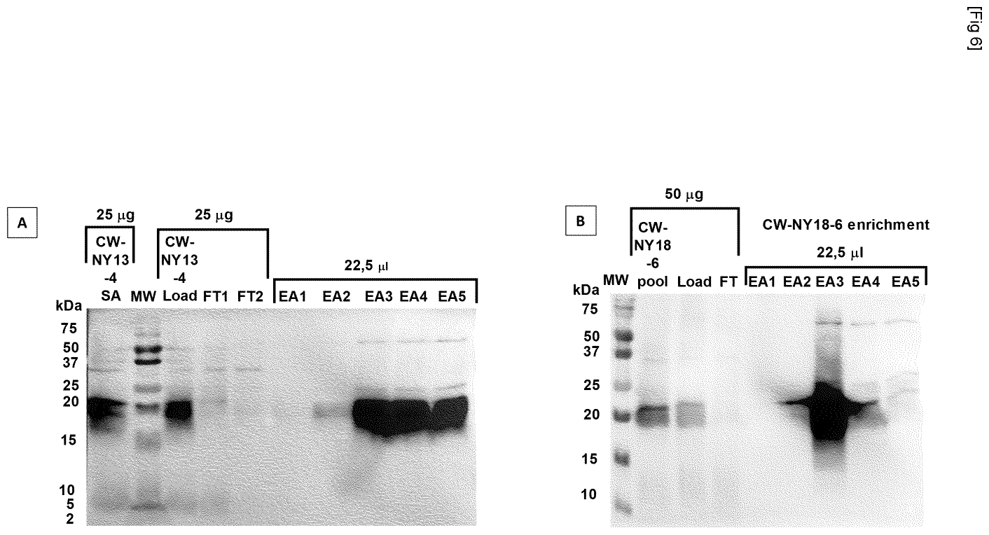

Fusion protein were purified by anti-Flag M2 affinity chromatography. Elution fractions were analysed by Western Blot analysis. The results, shown in FIG. 6, revealed the effectiveness of the affinity chromatography purification of the fusion proteins produced in the transformants CW-NY13-4 and CW-NY18-6.

The elution fraction from affinity chromatography containing the fusion protein 3F-FX-APRO-LGM-EK-NY2 or 3F-FX-APRO-LGM-TV-(NY3b)×5 were dialyzed in dialysis cassettes (3.5 kDa MWCO) against the buffer used in the next step of protease digestion.and concentrated using centrifugal concentrators (3 kDa MWCO).

Then the cleavage of the peptide or polypeptide from the carrier was performed with a site specific proteolysis of the fusion protein APRO-LGM-EK-NY2 or APRO-LGM-TV-(NY3b)×5 using enterokinase or TEV protease, respectively.

After an overnight incubation at 4° C., the digestions of each fusion protein were injected on a HiLoad 26/600 Superdex 30 prep grade column and run at a rate of 2.6 mL/min. These size exclusion chromatography (SEC) experiments allowed the purification of the peptide NY2 and polypeptide (NY3b)×5.

Further analysis on the SEC purified polypeptide (NY3b)×5 and peptide NY2 were performed by high performance liquid chromatography (HPLC) on C18 and C4 large pore reverse phase columns with 215 nm UV detection and mass spectrometry.

In order to cleave the polypeptides (NY3b)×5 into peptides by endoproteinase, the SEC elution fractions were evaporated and dialyzed for salts removing and buffer changing, using a dialysis tube with a 1 kDa cutoff.

After digestion by the Glu-C endoproteinase of the dialyzed samples as described in the example 1, the released peptides were purified by a size exclusion chromatography.

1. A recombinant microalgae comprising a nucleic acid sequence encoding:
(a) a recombinant peptide of SEQ ID NO:1;
(b) a recombinant peptide, polypeptide or protein consisting in repeated units of SEQ ID NO:1; or
(c) a derivative of (a) or (b);
the nucleic acid sequence being located in the chloroplast genome of microalgae.

2. A method for producing:
(a) a recombinant peptide of SEQ ID NO:1;
(b) a recombinant peptide, polypeptide or protein consisting in repeated units of SEQ ID NO:1; or
(c) a derivative of (a) or (b);
in the chloroplast of microalgae, wherein the method comprises transformation of the chloroplast genome of microalgae with a nucleic acid sequence encoding the recombinant protein, polypeptide or peptide.

3. The method according to claim 2, comprising:
(i) providing a nucleic acid sequence encoding the recombinant protein, polypeptide or peptide;
(ii) introducing the nucleic acid sequence according to (i) into an expression vector which is capable of expressing the nucleic acid sequence in microalgae host cell;
(iii) transforming the chloroplast genome of microalgae host cell by the expression vector.

4. The method according to claim 3, further comprising:
(iv) identifying the transformed microalgae host cell;
(v) characterizing the microalgae host cell for the production of recombinant protein, polypeptide or peptide expressed from the nucleic acid sequence; and
(vi) extracting the recombinant protein, polypeptide or peptide; and optionally
(vii) purifying the recombinant protein, polypeptide or peptide.

5. The according to claim 3, wherein the expression vector also comprises at least one expression cassette, the at least one expression cassette comprising the nucleic acid sequence encoding the recombinant protein, polypeptide or peptide.

6. The recombinant microalgae according to claim 1, wherein the nucleic acid sequence encoding the protein, polypeptide or peptide is codon optimized for expression in the chloroplast genome of the microalgae host cell.

7. The recombinant microalgae according to claim 1, wherein the derivative of (a) or (b) consists in an amino acid sequence at least 80% identical to the amino acid sequence of the recombinant peptide of SEQ ID NO:1 or of the recombinant peptide, polypeptide or protein consisting in repeated units of SEQ ID NO:1.

8. The recombinant microalgae according to claim 1, wherein the nucleic acid sequence encoding a recombinant protein, polypeptide or peptide is fused operationally at its 5′ or 3′ end to a nucleic acid sequence encoding a carrier.

9. The recombinant microalgae according to claim 1 wherein the nucleic acid sequence encoding a recombinant protein, polypeptide or peptide is operably linked to at least one regulatory sequence chosen from the psbD promoter and 5′UTR or the 16S rRNA promoter (Prrn) promoter fused with the atpA 5′UTR, the psaA promoter and 5′UTR, the atpA promoter and 5′ UTR, the atpA and rbcL 3′UTRs.

10. The method according to claim 5, wherein the at least one expression cassette further comprises a nucleic acid sequence encoding an epitope Tag peptide fused operationally at its 5′ or 3′end to the nucleic acid sequence encoding the recombinant protein, polypeptide or peptide.

11. The method according to claim 5 wherein the at least one expression cassette further comprises a nucleic acid sequence encoding a signal peptide.

12. The method according to claim 5, wherein the at least one expression cassette further comprises a nucleic acid sequence encoding an amino acid sequence allowing the production of the recombinant protein, polypeptide or peptide in specific cell compartment.

13. The recombinant microalgae according to claim 1, wherein the microalgae is selected from the group consisting of Chlorophyta, Chlorophyceae, Pleurastrophyceae, Prasinophyceae, Chromophyta, Bacillariophyceae, Chrysophyceae, Phaeophyceae, Eustigmatophyceae, Haptophyceae, Raphidophyceae, Xanthophyceae, Cryptophyta, Cryptophyceae, Rhodophyta, Porphyridiophycea, Stramenopiles, Glaucophyta, Glaucocystophyceae, Chlorarachniophyceae, Haptophyceae, Dinophyceae, Scenedesmaceae, Euglenophyta, Euglenophyceae.

14. The recombinant microalgae or method according to claim 13, wherein the microalgae is selected from the group consisting of Chlamydomonas, Chlorella, Dunaliella, Haematococcus, diatoms, Scenedesmaceae, Tetraselmis, Ostreococcus, Porphyridium, and Nannochloropsis.

15. A method for manufacturing a cosmetic composition, comprising adding as a component the peptide and/or polypeptide and/or protein aqueous mixture produced according to the method of claim 2.

16. A cosmetic non-therapeutical treatment comprising as a component the peptide and/or polypeptide and/or protein aqueous mixture produced according to the method of claim 2 and free of algae debris.

17. (canceled)


SEQUENCE LISTING (81 sequences)

SEQ ID NO: 1 - protein, 5 aa, Artificial Sequence
  NY2 - KTTKS
KTTKS

SEQ ID NO: 2 - protein, 6 aa, Artificial Sequence
  GNY2- GKTTKS- derivative of KTTKS
GKTTKS

SEQ ID NO: 3 - protein, 6 aa, Artificial Sequence
  NY3a- derivative of KTTKS
KTTKSD

SEQ ID NO: 4 - protein, 6 aa, Artificial Sequence
  NY3b - derivative of KTTKS
KTTKSE

SEQ ID NO: 5 - protein, 6 aa, Artificial Sequence
  peptiide - derivative of KTTKS
DKTTKS

SEQ ID NO: 6 - protein, 6 aa, Artificial Sequence
  peptide - derivative of KTTKS
EKTTKS

SEQ ID NO: 7 - protein, 7 aa, Artificial Sequence
  peptide - derivative of GKTTKS
GKTTKSD

SEQ ID NO: 8 - protein, 7 aa, Artificial Sequence
  peptide - derivative of GKTTKS
GKTTKSE

SEQ ID NO: 9 - protein, 30 aa, Artificial Sequence
  (NY3a)x5 - polypepttde derivative
KTTKSDKTTKSDKTTKSDKTTKSDKTTKSD

SEQ ID NO: 10 - protein, 31 aa, Artificial Sequence
  G((NY3a)x5)- polypeptide derivative
GKTTKSDKTTKSDKTTKSDKTTKSDKTTKSD

SEQ ID NO: 11 - protein, 30 aa, Artificial Sequence
  (NY3b)x5 - polypeptide derivative
KTTKSEKTTKSEKTTKSEKTTKSEKTTKSE

SEQ ID NO: 12 - protein, 31 aa, Artificial Sequence
  G((NY3b)x5- polypeptide derivative
GKTTKSEKTTKSEKTTKSEKTTKSEKTTKSE

SEQ ID NO: 13 - DNA, 15 bp, Artificial Sequence
  NY2- ADN coding for KTTKS
aaaacaactaaatca

SEQ ID NO: 14 - DNA, 18 bp, Artificial Sequence
  GNY2- GKTTKS- ADN coding for derivative of KTTKS
ggtaaaacaactaaatca

SEQ ID NO: 15 - DNA, 90 bp, Artificial Sequence
  (NY3a)x5- ADN coding for polypeptide derivative
aagactaccaaaagtgataaaacaactaaaagcgataagacaactaaatctgataaaacaactaaatcagacaaaacaacaaaatcagat

SEQ ID NO: 16 - DNA, 93 bp, Artificial Sequence
  G(NY3a)x5- ADN coding for polypeptide derivative
ggtaagactaccaaaagtgataaaacaactaaaagcgataagacaactaaatctgataaaacaactaaatcagacaaaacaacaaaatcagat

SEQ ID NO: 17 - DNA, 90 bp, Artificial Sequence
  (NY3b)x5 - ADN coding for polypeptide derivative
aaaacaactaaatcagaaaaaactacaaaaagtgagaagactacaaaatctgagaaaacaaccaagtcagaaaagacaacaaaatcagaa

SEQ ID NO: 18 - DNA, 93 bp, Artificial Sequence
  G(NY3b)x5 - ADN coding for polypeptide derivative
ggtaaaacaactaaatcagaaaaaactacaaaaagtgagaagactacaaaatctgagaaaacaaccaagtcagaaaagacaacaaaatcagaa

SEQ ID NO: 19 - protein, 8 aa, Artificial Sequence
  Flag Tag
DYKDDDDK

SEQ ID NO: 20 - protein, 24 aa, Artificial Sequence
  3xFlag Tag
DYKDDDDKDYKDDDDKDYKDDDDK

SEQ ID NO: 21 - protein, 9 aa, Artificial Sequence
  HA Tag
YPYDVPDYA

SEQ ID NO: 22 - protein, 27 aa, Artificial Sequence
  3xHA Tag
YPYDVPDYAYPYDVPDYAYPYDVPDYA

SEQ ID NO: 23 - protein, 6 aa, Artificial Sequence
  His Tag
HHHHHH

SEQ ID NO: 24 - protein, 41 aa, Artificial Sequence
  SP
NNNDLFQASRRRFLAQLGGLTVAGMLGPSLLTPRRATAAQA

SEQ ID NO: 25 - protein, 58 aa, Bos taurus [sequence omitted]

SEQ ID NO: 26 - protein, 140 aa, Artificial Sequence
  HA-SP-3F-FX-APRO
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGA

SEQ ID NO: 27 - protein, 423 aa, Artificial Sequence
  ADN coding for HA-SP-3F-FX-APRO
ATGGCATATCCTTATGATGTACCAGATTATGCCAATAATAACGACTTATTTCAAGCATCACGTCGTCGTTTCTTAGCTCAATTAGGAGGTTTAACAGTTGCTGGTATGTTAGGTCCATCTTTACTTACTCCACGTAGAGCTACAGCAGCTCAAGCTGATTACAAAGATGATGACGATAAAGACTATAAAGATGATGATGACAAAGATTACAAAGACGATGATGATAAAGGATCCATTGAAGGTCGTAGACCAGACTTTTGCTTAGAACCACCATATACAGGTCCATGTAAAGCTCGTATCATTCGCTATTTCTACAATGCAAAAGCAGGATTATGTCAAACATTTGTGTATGGAGGTTGTCGTGCTAAACGTAACAACTTTAAATCAGCTGAAGATTGTATGCGTACTTGTGGTGGTGCTTAA

SEQ ID NO: 28 - protein, 82 aa, Artificial Sequence
  3F-APRO
DYKDDDDKDYKDDDDKDYKDDDDKRPDFCLEPPYTGPCKARIIRYFYNAKAGLCQTFVYGGCRAKRNNFKSAEDCMRTCGGA

SEQ ID NO: 29 - protein, 88 aa, Artificial Sequence
  3F-FX-APRO
DYKDDDDKDYKDDDDKDYKDDDDKGSIEGRRPDFCLEPPYTGPCKARIIRYFYNAKAGLCQTFVYGGCRAKRNNFKSAEDCMRTCGGA

SEQ ID NO: 30 - protein, 14 aa, Artificial Sequence
  Linker LG
RSGGGGSGGGGSGS

SEQ ID NO: 31 - protein, 18 aa, Artificial Sequence
  Linker LGM
RSGGGGSSGGGGGGSSRS

SEQ ID NO: 32 - protein, 7 aa, Artificial Sequence
  TEV protease TV
ENLYFQG

SEQ ID NO: 33 - protein, 5 aa, Artificial Sequence
  enterokinase EK
DDDDK

SEQ ID NO: 34 - protein, 168 aa, Artificial Sequence
  HA-SP-3F-FX-APRO-LGM-EK-NY2
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKKTTKS

SEQ ID NO: 35 - DNA, 507 bp, Artificial Sequence
  DNA coding for HA-SP-3F-FX-APRO-LGM-EK-NY2
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgacgatgacgacaagaaaacaactaaatcataa

SEQ ID NO: 36 - protein, 170 aa, Artificial Sequence
  HA-SP-3F-FX-APRO-LGM-TV-NY2
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKS

SEQ ID NO: 37 - DNA, 513 bp, Artificial Sequence
  DNA coding for HA-SP-3F-FX-APRO-LGM-TV-NY2
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgaaaatttatattttcaaggtaaaacaactaaatcataa

SEQ ID NO: 38 - protein, 194 aa, Artificial Sequence
  HA-SP-3F-FX-APRO-LGM-EK-(NY3a)x5
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKGKTTKSDKTTKSDKTTKSDKTTKSDKTTKSD

SEQ ID NO: 39 - DNA, 585 bp, Artificial Sequence
  DNA coding for HA-SP-3F-FX-APRO-LGM-EK-(NY3a)x5
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgacgatgacgacaagggtaagactaccaaaagtgataaaacaactaaaagcgataagacaactaaatctgataaaacaactaaatcagacaaaacaacaaaatcagattaa

SEQ ID NO: 40 - protein, 195 aa, Artificial Sequence
  HA-SP-3F-FX-APRO-LGM-TV-(NY3a)x5
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKSDKTTKSDKTTKSDKTTKSDKTTKSD

SEQ ID NO: 41 - DNA, 588 bp, Artificial Sequence
  DNA coding for HA-SP-3F-FX-APRO-LGM-TV-(NY3a)x5
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgaaaatttatattttcaaggtaagactaccaaaagtgataaaacaactaaaagcgataagacaactaaatctgataaaacaactaaatcagacaaaacaacaaaatcagattaa

SEQ ID NO: 42 - protein, 194 aa, Artificial Sequence
  HA-SP-3F-FX-APRO-LGM-EK-(NY3b)x5
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKGKTTKSEKTTKSEKTTKSEKTTKSEKTTKSE

SEQ ID NO: 43 - DNA, 585 bp, Artificial Sequence
  DNA coding for HA-SP-3F-FX-APRO-LGM-EK-(NY3b)x5
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgacgatgacgacaagggtaaaacaactaaatcagaaaaaactacaaaaagtgagaagactacaaaatctgagaaaacaaccaagtcagaaaagacaacaaaatcagaataa

SEQ ID NO: 44 - protein, 195 aa, Artificial Sequence
  HA-SP-3F-FX-APRO-LGM-TV-(NY3b)x5
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKSEKTTKSEKTTKSEKTTKSEKTTKSE

SEQ ID NO: 45 - DNA, 588 bp, Artificial Sequence
  DNA coding for HA-SP-3F-FX-APRO-LGM-TV-(NY3b)x5
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgaaaatttatattttcaaggtaaaacaactaaatcagaaaaaactacaaaaagtgagaagactacaaaatctgagaaaacaaccaagtcagaaaagacaacaaaatcagaataa

SEQ ID NO: 46 - protein, 4 aa, Artificial Sequence
  tetrapeptide
GQPR

SEQ ID NO: 47 - protein, 4 aa, Artificial Sequence
  tetrapeptide
TKPR

SEQ ID NO: 48 - protein, 4 aa, Artificial Sequence
  tetrapeptide
KTFK

SEQ ID NO: 49 - protein, 4 aa, Artificial Sequence
  tetrapeptide
KTAK

SEQ ID NO: 50 - protein, 4 aa, Artificial Sequence
  tetrapeptide
KAYK

SEQ ID NO: 51 - protein, 4 aa, Artificial Sequence
  tetrapeptide
KFYK

SEQ ID NO: 52 - protein, 4 aa, Artificial Sequence
  KAvaMO2K
  MISC_FEATURE (2)..(2): Ava (5 amino valeric acid)
  MISC_FEATURE (3)..(3): MO2 sulfoxide methionine
KXXK

SEQ ID NO: 53 - protein, 4 aa, Artificial Sequence
  tetrapeptide
  MISC_FEATURE (2)..(3): MO2 (sulfoxide methionine)
KXTK

SEQ ID NO: 54 - protein, 4 aa, Artificial Sequence
  tetrapeptide
AQPR

SEQ ID NO: 55 - protein, 4 aa, Artificial Sequence
  tetrapeptide
AQPK

SEQ ID NO: 56 - protein, 5 aa, Artificial Sequence
  pentapeptide
YGGFL

SEQ ID NO: 57 - protein, 5 aa, Artificial Sequence
  pentapeptide
YGGFP

SEQ ID NO: 58 - protein, 6 aa, Artificial Sequence
  hexapeptide
HLDIIW

SEQ ID NO: 59 - protein, 6 aa, Artificial Sequence
  hexapeptide
HLDIIF

SEQ ID NO: 60 - protein, 6 aa, Artificial Sequence
  hexapeptide
  MISC_FEATURE (6)..(6): Tpi (tryptoline-3-carboxylic acid)
HLDIIX

SEQ ID NO: 61 - protein, 4 aa, Artificial Sequence
  Factor Xa FX
IEGR

SEQ ID NO: 62 - protein, 143 aa, Artificial Sequence
  3F-FX-APRO-LGM-TV-(NY3b)x5
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKSEKTTKSEKTTKSEKTTKSEKTTKSE

SEQ ID NO: 63 - protein, 142 aa, Artificial Sequence
  3F-FX-APRO-LGM-EK-(NY3b)x5
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKGKTTKSEKTTKSEKTTKSEKTTKSEKTTKSE

SEQ ID NO: 64 - protein, 143 aa, Artificial Sequence
  3F-FX-APRO-LGM-TV-(NY3a)x5
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKSDKTTKSDKTTKSDKTTKSDKTTKSD

SEQ ID NO: 65 - protein, 142 aa, Artificial Sequence
  3F-FX-APRO-LGM-EK-(NY3a)x5
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKGKTTKSDKTTKSDKTTKSDKTTKSDKTTKSD

SEQ ID NO: 66 - protein, 116 aa, Artificial Sequence
  3F-FX-APRO-LGM-EK-NY2
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKKTTKS

SEQ ID NO: 67 - protein, 118 aa, Artificial Sequence
  3F-FX-APRO-LGM-TV-NY2
DYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGKTTKS

SEQ ID NO: 68 - DNA, 237 bp, Artificial Sequence
  GNC-LENY3a2
cagctgaagattgtatgcgtacttgtggtggtgctagatctggtggaggcggttcaagtggtggaggaggtggcggatcttcaagatctgacgatgacgacaagggtaagactaccaaaagtgataaaacaactaaaagcgataagacaactaaatctgataaaacaactaaatcagacaaaacaacaaaatcagattaagtttaaaccaggtgacctgcagagctagctactgcag

SEQ ID NO: 69 - DNA, 237 bp, Artificial Sequence
  GNC-LENY3b1
cagctgaagattgtatgcgtacttgtggtggtgctagatctggtggaggcggttcaagtggtggaggaggtggcggatcttcaagatctgacgatgacgacaagggtaaaacaactaaatcagaaaaaactacaaaaagtgagaagactacaaaatctgagaaaacaaccaagtcagaaaagacaacaaaatcagaataagtttaaaccaggtgacctgcagagctagctactgcag

SEQ ID NO: 70 - DNA, 240 bp, Artificial Sequence
  GNC-LTNY3a2
cagctgaagattgtatgcgtacttgtggtggtgctagatctggtggaggcggttcaagtggtggaggaggtggcggatcttcaagatctgaaaatttatattttcaaggtaagactaccaaaagtgataaaacaactaaaagcgataagacaactaaatctgataaaacaactaaatcagacaaaacaacaaaatcagattaagtttaaaccaggtgacctgcagagctagctactgcag

SEQ ID NO: 71 - DNA, 240 bp, Artificial Sequence
  GNC-LTNY3b1
cagctgaagattgtatgcgtacttgtggtggtgctagatctggtggaggcggttcaagtggtggaggaggtggcggatcttcaagatctgaaaatttatattttcaaggtaaaacaactaaatcagaaaaaactacaaaaagtgagaagactacaaaatctgagaaaacaaccaagtcagaaaagacaacaaaatcagaataagtttaaaccaggtgacctgcagagctagctactgcag

SEQ ID NO: 72 - DNA, 355 bp, Artificial Sequence
  GNC-ALENY2
gatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgacgatgacgacaagaaaacaactaaatcataagtttaaaccaggtgacctgcagagctagctactgcag

SEQ ID NO: 73 - DNA, 361 bp, Artificial Sequence
  GNC-ALTNY2
gatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctagatctggtggaggcggttcaagtggtggaggaggtggcggatcttcaagatctgaaaatttatattttcaaggtaaaacaactaaatcataagtttaaaccaggtgacctgcagagctagctactgcag

SEQ ID NO: 74 - DNA, 35 bp, Artificial Sequence
  primer
cctacttaattaaaaactgcagtagctagctctgc

SEQ ID NO: 75 - DNA, 34 bp, Artificial Sequence
  primer
cgatgagttgtttttttattttggagatacacgc

SEQ ID NO: 76 - DNA, 1025 bp, Artificial Sequence
  Prrn-5′UTRatpA
tttttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacagactagtcagactcggggggcaggcaacaaatttatttattgtcccgtaaggggaaggggaaaacaattattattttactgcggagcagcttgttattgaaattttattaaaaaaaaaataaaaatttgacaaaaaaaaataaaaaagttaaattaaaaacactgggaatgttctacatcataaaaatcaaaagggtttaaaatcccgacaaaatttaaactttaaagagtggcgcctaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttctttttt

SEQ ID NO: 77 - DNA, 435 bp, Artificial Sequence
  3′UTRrbcL
aagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatactaaagcggatgtaactcaatcggtagagtgcgatcc

SEQ ID NO: 78 - DNA, 424 bp, Artificial Sequence
  PpsbD-5′UTR
atgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcacc

SEQ ID NO: 79 - DNA, 400 bp, Artificial Sequence
  3′UTRatpA
tttttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacag

SEQ ID NO: 80 - DNA, 123 bp, Artificial Sequence
  DNA coding SP
aataataacgacttatttcaagcatcacgtcgtcgtttcttagctcaattaggaggtttaacagttgctggtatgttaggtccatctttacttactccacgtagagctacagcagctcaagct

SEQ ID NO: 81 - DNA, 174 bp, Bos taurus
cggcctgacttctgcctagagcctccatatacgggtccctgcaaggccagaattatcagatacttctacaacgccaaggctgggctctgccagacctttgtatatggcggctgcagagctaaaagaaacaatttcaagagcgcagaggactgcatgaggacctgtggtggtgct